(19) United States
(12) Patent Application Publication
(10) Pub. No.: US 2026/0155247 A1
(43) Pub. Date: Jun. 4, 2026

(54) LARGE LANGUAGE MODEL-BASED NEUROSTIMULATION PROGRAMMING RECOMMENDATIONS

(71) Applicant: Boston Scientific Neuromodulation Corporation, Valencia, CA (US)

(72) Inventors: Bradley Lawrence Hershey, Carrollton, TX (US); G. Karl Steinke, Valencia, CA (US); Lisa Denise Moore, Glendale, CA (US)

(73) Assignee: Boston Scientific Neuromodulation Corporation, Valencia, CA (US)

(21) Appl. No.: 19/407,877

(22) Filed: Dec. 3, 2025

Related U.S. Application Data
(60) Provisional application No. 63/727,926, filed on Dec. 4, 2024.

(51) Int. Cl. G16H 40/67; A61N 1/372; G16H 50/20

(52) U.S. Cl. CPC G16H 40/67; A61N 1/37235; G16H 50/20

(57) ABSTRACT

This disclosure relates to generating large language model (LLM)-based neurostimulation programming recommendations. An example method for large language model (LLM)-based neurostimulation programming recommendations comprises receiving patient inputs from a patient undergoing neurostimulation from a programmed neurostimulation device, programmer input data related to the programmed neurostimulation device, or both; querying a trained LLM with the patient inputs, the programmer input data, or both, to generate a programming recommendation, a recommended action, or both; providing a representation of the programming recommendation, the recommended action, or both; receiving an input to initiate the programming recommendation, the recommended action, or both; and initiating an action for the neurostimulation treatment, based on the input to initiate the programming recommendation, the recommended action, or both.

### CROSS REFERENCE TO RELATED APPLICATIONS

The present application claims the benefit of U.S. Provisional Patent Application Ser. No. 63/727,926, filed Dec. 4, 2024, the disclosure of which is incorporated herein by reference.

### TECHNICAL FIELD

The present disclosure relates generally to medical devices, and more particularly, to systems, devices, and methods for determining electrical stimulation programming recommendations by querying a large language model (LLM) with programmer inputs, patient inputs, or both.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver therapy. An implantable neurostimulation system may include an implantable electrical neurostimulator, also referred to as an implantable pulse generator (IPG), and one or more implantable leads each including one or more electrodes. The implantable electrical neurostimulator delivers neurostimulation energy through one or more electrodes placed on or near a target site in the nervous system.

A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to create an analgesic effect that masks pain sensation. While modern electronics can accommodate the need for generating and delivering stimulation energy in a variety of forms, the capability of a neurostimulation system depends on its post-manufacturing programmability to a great extent. For example, a sophisticated neurostimulation program may only provide meaningful benefits to a patient when the neurostimulation program is customized (e.g., via programming) for a particular patient.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

As used herein, the terms “neurostimulator,” “stimulator,” “neurostimulation,” and “stimulation” generally refer to the delivery of electrical energy that affects the neuronal activity of neural tissue, which may be excitatory or inhibitory; for example, by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

The programming of neurostimulation devices involves complex interactions between anatomy, neurophysiology, and electric fields. Traditionally, subject matter experts with specialized training in these areas create stimulation programs by adjusting multiple parameters to achieve optimal therapeutic outcomes. The process typically involves iterative adjustments based on patient feedback and clinical observations. Hence, some previous approaches to neurostimulation programming rely heavily on subject matter experts with specialized training in anatomy, neurophysiology, and electric field interactions. These experts would manually adjust stimulation parameters based on patient feedback during programming sessions. This process was time-consuming, inconsistent across programmers, and limited by the individual programmer's experience. Additionally, although some neurostimulation devices provide the capability to permit a patient to switch between programs or change the level of a certain stimulation effect, it is often unclear whether such changes or which changes may be beneficial to a patient and result in improvement (e.g., a reduction in pain) to the patient's medical condition. Moreover, some approaches may attempt to employ various natural language models to assess patient sentiment and/or the efficacy of neurostimulation programming via text. However, such approaches may be prone to inaccuracies (e.g., due to the type and/or relatively small size of the training datasets and/or due a reliance on text specific analysis) and/or otherwise may be ineffective.

Recent advancements in artificial intelligence and machine learning have opened new possibilities for enhancing the programming process and improving treatment efficacy. As such, the systems, devices, and methods herein utilize a large language model (LLM) (e.g., that is trained at least with proprietary expert knowledge in neuromodulation) to assist in neurostimulation programming, as detailed herein. The proprietary expert knowledge, as detailed herein, can include various neuromodulation programming goals (e.g., from the perspective of the programmer and/or patient) and/or various mechanisms (e.g., specific programming settings or programming weights/nudges to achieve the neuromodulation programming goals), etc. For example, the systems, devices, and methods herein can receive patient input data and programmer input data, process the data (or a derivative of the data) using a LLM (e.g., via querying the LLM), and generate programming recommendations and/or recommended actions (e.g., generate additional questions configured to elicit further information pertaining to pain or other real-world feedback from the patient, etc.). These recommendations can be automatically applied or can be provided as an audio output and/or a video output presented via a display to one or both of the patient and a programmer as a recommendation. In any case, once the recommendations are applied to the neurostimulation device, additional feedback can be collected to continue to iteratively adjust the neurostimulation programming (e.g., provide subsequent programming recommendations) until a desired outcome is achieved (e.g., from the perspective of the programmer and/or patient). In some embodiments, the system, devices, and methods herein can determine when a desired outcome is achieved through various mechanisms, including real-time patient feedback (e.g., real-time patient feedback provided by voice e.g., to a programmer, real-time patient feedback provided via free form text to a chat-bot, etc.), programmer assessment, and/or analysis of objective metrics from the neurostimulation device, among other possibilities such as based on other information obtained from other devices and/or applications (e.g., various phone and/or web based applications) and/or portals (e.g., healthcare or provider portals), etc.

In some embodiments, the system, devices, and methods herein can utilize custom training of the LLM. For instance, the LLM and/or another system such as those described herein can be trained with subject matter expert input and/or various patient input (e.g., programmer-patient interaction data). For instance, the LLM can be trained (e.g., at least is initially trained to minimum viable state) with subject matter expert input. Such subject matter expert input can be based at least in part on or exclusively on proprietary data sources which can promote data security and enhanced accuracy of the LLM outputs (e.g., enhanced programming recommendations). In some embodiments, the LLM can be trained (or retrained) on a data set that includes a plurality of past patient interactions (e.g., locally or globally). For instance, in some embodiments, the methods herein can train an LLM at least on a data set that includes data indicative of a plurality of past patient interactions that are local to one or more current neurostimulation patient (e.g., within the same state, same country, same practice, same training family tree (including schools, degrees, or mentors), same manufacturer sales territory, etc. as one or more current neurostimulation patient patient). Employing local data for training can improve an accuracy of the outputs of the trained model, for instance, by accounting for local customs and/or treat methodologies. In some embodiments, the LLM can be trained (e.g., retrained) periodically or continuously. For example, the LLM can be retrained based on outcomes associated with programming recommendations that are applied to a neurostimulation device (e.g., based on additional feedback can be collected to continue to iteratively adjust the programming until a desired outcome is achieved and/or can be retrained based on incorporation of new program types (new stimulation patterns, etc.) and/or published literature (e.g., based on newly published scientific literature that is proprietary and/or publicly available). In some embodiments, the LLM can aid in creating and/or updating a programming profile. For instance, the LLM can update a programming profile (e.g., locally) after each programming session, via a web portal, and/or after a quantity of programming sessions. In some embodiments, the LLM and/or feedback mechanisms as described herein can collate programming information and feedback from a plurality of programming sessions to generate an aggregated feedback data set that is weighted (e.g., with weights of 20/30/50, etc. corresponding to three programming sessions) and hence provides an average or aggregated representation of feedback. Moreover, in some embodiments, the systems, devices, and methods herein can be configured to provide programming recommendations that are within a predefined range and/or satisfy a threshold (e.g., comply with a maximum permissible amount duration and/or a maximum amplitude of stimulation, etc.) to ensure that the programming recommendations are compliant with known safety considerations and/or clinician-approved settings of the neurostimulation device. The programming recommendations provided can be specific to a particular patient (e.g., based on real-time feedback received from the particular patient), patient group (e.g., indication or pain type or patient phenotype or group, such as age or current or desired activity level) and/or can be specific to a particular type of neurostimulation device or therapy such as being specific to SCS or DBS, among other types of neurostimulation therapies. In some embodiments, the programming recommendations can be localized programming recommendations such as those that are local to the patient and/or the programmer associated with the patient. In some embodiments, the programming recommendations can be disease or condition specific. For instance, SCS or DBS can be utilized to treat various diseases or conditions and therefore providing recommendations that are specific to both SCS or DBS in addition to one or more diseases or conditions being treated can provide enhanced (e.g., more clinically effective) recommendations as compared to some other approaches.

In some embodiments the recommendations can be provided periodically (e.g., at a fixed time interval e.g., such as once a day or once every few hours) and/or can be provided responsive to an input (e.g., responsive to an input from a programmer, physician, and/or patient. Alternatively, or in addition, in some embodiments the recommendations can be provided responsive to satisfying a change threshold. For instance, small recommend changes in amplitude may not be provided as a recommendation (e.g., may not satisfy a threshold e.g., 10 percent change from a currently applied programming setting), while larger recommended changes such as changing a stimulation program, changing a quantity or using different electrodes, and/or making a large (e.g., greater than 10 percent change) in a currently applied programming setting may satisfy the threshold and thus may be communicated as a recommendation.

As mentioned, in some embodiments, the LLM can be configured to provide outputs (e.g., recommended actions) in the form of directives, suggestions, observations, or questions to guide the programmer and/or patient. That is, the recommended action can correspond to actions other than specific parameter adjustments. Examples of recommend actions include, but are not limited to, recommending actions for the patient and/or actions for the programmer to take. Examples of actions for the patient to take include changing posture, taking a break, taking a medication, taking a set of actions (stand, walk, sit), responding to a question, and/or other recommended actions such as those described herein. Examples of actions for the programming to take including generalized programming recommendations (e.g., that are not at the level of specificity of the programming parameters) such as changing a therapy type (sub-perception versus paresthesia based, for example), changing dosing provided to the patient (increasing or decreasing any of amplitude, pulse-width, and rate, for example), alter scheduling associated with the neurostimulation device (e.g., to turn on or off stimulation or alter a timing /cheduling of the stimulation, and/or, advice for the programmer to provide to the patient or any person in a care team associated with the patient—e.g., when and how to switch between programs while sleeping, active, or otherwise, among other recommended actions such as those described herein. Other types of recommendations such as those described herein (e.g., changing a patient's medication dose, altering a patient's physical position, etc.) are possible.

The subsequent responses e.g., from the programmer and/or patient can be factored into a subsequent LLM-based programming recommendations. Hence, the approaches herein provide an additional type of input (e.g., responsive to LLM based questions, suggestions, etc.) that can better tailor subsequent LLM-based programming recommendations to a particular user/use case, as compared to other approaches such as those the rely solely on a patient and/or programmer input (e.g., text analysis of patient statements) in the absence of such supplemental information.

In view of the above, the systems, devices, and methods herein yield significant value at least through increased consistency in programming across different users (e.g., different patients and/or different programmers, different geographies, etc.), improved efficiency in achieving a desired outcome, and/or better overall results/effectiveness (e.g., from the perspective of a patient and/or programmer), by leveraging a broader knowledge base as compared to individual programmer experience, leveraging real-time patient feedback (e.g., feedback such as audio, text, and/or video feedback obtained during a communication session with a chatbot and/or during a communication session between a patient-programmer), and/or leveraging additional information provided responsive to recommended actions (e.g., questions or suggestions) from the LLM.

In one example, a system for generating large language model (LLM)-based neurostimulation programming recommendations is provided. The system comprising: a programmed neurostimulation device; a large language model (LLM) trained with expert knowledge in neurostimulation programming; and a processor configured to: receive patient inputs from a patient undergoing neurostimulation from a programmed neurostimulation device, programmer input data related to the programmed neurostimulation device, or both; query a trained LLM with the patient inputs, the programmer input data, or both, to generate a programming recommendation, a recommended action, or both; provide, via a device, a representation of the programming recommendation, the recommended action, or both, wherein the device is a clinician interaction computing device, a patient interaction computing device, or both; receiving an input, via the device, to initiate the programming recommendation, the recommended action, or both; and responsive to receipt of the input, initiate an action for the neurostimulation treatment, based on the input to initiate the programming recommendation, the recommended action, or both.

In some aspects, wherein the programmed neurostimulation device is a spinal cord stimulation device.

In some aspects, wherein the programmed neurostimulation device is a deep brain stimulation device.

In some aspects, wherein the input comprises an input to initiate the programming recommendation; and wherein initiating the action comprises applying the programming recommendation to the programmed neurostimulation device, and wherein the processor is further configured to: receive patient feedback associated with the applied programming recommendation, receiving programmer feedback associated with the applied programming recommendation, or both; and retrain the trained LLM based on the received patient feedback associated with the applied programming recommendation, the programmer feedback associated with the applied programming recommendation, or both.

In some aspects, wherein the processor is further configured to: receive updated programmer input data, updated patient input, or both; query the retrained LLM with the updated programmer input data, the updated patient input, or both, to generate an updated programming recommendation, an updated recommended action, or both; provide, via the device, the updated programming recommendation, the updated recommended action, or both; receive an input to initiate the updated programming recommendation, the updated recommended action, or both; and initiate an updated action for the neurostimulation treatment, based on the input to initiate the updated programming recommendation, the updated recommended action, or both.

In some aspects, wherein the LLM is trained with at least neuromodulation subject matter expert input, and wherein the processor is further configured to retrain the trained LLM with the patient inputs from a patient undergoing neurostimulation, the programmer input data related to the programmed neurostimulation device, or both.

In some aspects, wherein the processor is further configured to retrain the trained LLM based on programmer-patient interaction data obtained during a programmer-patient interaction, wherein the programmer-patient interaction data further comprises voice-to-text data, voice-to-voice data, and/or visual data associated with the patient obtained during the programmer-patient interaction.

In some aspects, wherein the programming recommendation is based on predefined neurostimulation parameters in the trained LLM, the predefined neurostimulation parameters including: available programming settings for the programmed neurostimulation device; clinician approved programming settings for the programmed neurostimulation device; previously utilized programming settings for the programmed neurostimulation device; or any combination thereof.

In some aspects, wherein the programming recommendation further comprises a recommendation to apply a different neurostimulation program to the programmed neurostimulation device than a currently applied neurostimulation program to the programmed neurostimulation device, and wherein initiating the action for the neurostimulation treatment further comprises providing the programming recommendation to apply the different neurostimulation program to a clinician interaction computing device, a patient interaction computing device, or both.

In some aspects, wherein initiation of the action for the neurostimulation treatment further comprises providing a question to the patient related to the neurostimulation treatment, providing a question to the programmer related to the neurostimulation treatment, or both.

In some aspects, wherein the trained LLM further comprises a trained LLM that is trained at least on a data set that includes training on a plurality of past patient interactions that are local to the patient.

In some aspects, wherein the query of the trained LLM is configured to generate both the programming recommendation and the recommended action.

In some aspects, wherein the recommended action further comprises a notification that is indicative at least of a scheduled time of application of the programming recommendation to the programmed neurostimulation device, and wherein the processor is further configured to provide the notification to the patient.

In another aspects, a non-transitory computer-readable medium is provided. The medium comprising instructions that, when executed by a processor, cause the processor to: receive two or more different types of real-time patient inputs from the patient that are related to a programmed neurostimulation device and programmer input data related to the programmed neurostimulation device, wherein the two or more different types of real-time patient inputs include two or more of text inputs, audio inputs, and visual inputs; query a trained large language model (LLM) with the plurality of different types of real-time patient inputs or a derivative of the plurality of real-time patient inputs and programmer input data, to generate a programming recommendation, a recommended action, or both; display, via a display of a device, a representation of the programming recommendation, the recommended action, or both; receive input to initiate the programming recommendation, the recommended action, or both; and responsive to receipt of the input, initiate an action for the neurostimulation treatment, based on the input to initiate the programming recommendation, the recommended action, or both.

In some aspects, wherein the query of the trained LLM generates both the programming recommendation and the recommended action, and wherein the recommended action further comprises providing a notification to the patient that is indicative of a scheduled time of application the programming recommendation to the programmed neurostimulation device; details associated with the programming recommendation; or both, and wherein the processor is further configured to provide the recommendation to the patient.

In another aspect a method for generating large language model (LLM)-based neurostimulation programming recommendations is provided. The method comprising: receiving patient inputs from a patient undergoing neurostimulation from a programmed neurostimulation device, programmer input data related to the programmed neurostimulation device, or both; querying a trained LLM with the patient inputs, the programmer input data, or both, to generate a programming recommendation, a recommended action, or both; providing, via a device, a representation of the programming recommendation, the recommended action, or both, wherein the device is a clinician interaction computing device, a patient interaction computing device, or both; receiving an input, via the device, to initiate the programming recommendation, the recommended action, or both; and responsive to receipt of the input, initiating an action for the neurostimulation treatment, based on the input to initiate the programming recommendation, the recommended action, or both.

In some aspects, wherein the input comprises an input to initiate the programming recommendation; and wherein initiating the action comprises applying the programming recommendation to the programmed neurostimulation device.

In some aspects, receiving patient feedback associated with the applied programming recommendation, receiving programmer feedback associated with the applied programming recommendation, or both; and retraining the trained LLM based on the received patient feedback associated with the applied programming recommendation, the programmer feedback associated with the applied programming recommendation, or both.

In some aspects, further comprising: receiving updated programmer input data, updated patient input, or both; querying the retrained LLM with the updated programmer input data, the updated patient input, or both, to generate an updated programming recommendation, an updated recommended action, or both; providing, via the device, the updated programming recommendation, the updated recommended action, or both; receiving an input to initiate the updated programming recommendation, the updated recommended action, or both; and initiating an updated action for the neurostimulation treatment, based on the input to initiate the updated programming recommendation, the updated recommended action, or both.

In some aspects, further comprising training a LLM to yield the trained LLM, wherein the LLM is trained with at least neuromodulation subject matter expert input.

In some aspects, wherein the patient inputs are provided during a communication session with a chatbot, during a communication session with a programmer, or both.

In some aspects, further comprising retraining the trained LLM with the patient inputs from a patient undergoing neurostimulation, the programmer input data related to the programmed neurostimulation device, or both.

In some aspects, further comprising retraining the trained LLM based on programmer-patient interaction data obtained during a programmer-patient interaction, wherein the programmer-patient interaction data further comprises voice-to-text data, voice-to-voice data, and/or visual data associated with the patient obtained during the programmer-patient interaction.

In some aspects, further comprising generating, via the trained LLM, a programming recommendation, wherein the programming recommendation is based on predefined neurostimulation parameters in the trained LLM, the predefined neurostimulation parameters including: available programming settings for the programmed neurostimulation device; clinician approved programming settings for the programmed neurostimulation device; previously utilized programming settings for the programmed neurostimulation device; or any combination thereof.

In some aspects, wherein the programming recommendation further comprises a recommendation to modify a parameter of a currently applied neurostimulation program to the programmed neurostimulation device, and wherein initiating the action for the neurostimulation treatment further comprises providing the programming recommendation to modify a parameter to a clinician interaction computing device, a patient interaction computing device, or both.

In some aspects, wherein the programming recommendation further comprises a recommendation to apply a different neurostimulation program to the programmed neurostimulation device than a currently applied neurostimulation program to the programmed neurostimulation device, and wherein initiating the action for the neurostimulation treatment further comprises providing the programming recommendation to apply the different neurostimulation program to a clinician interaction computing device, a patient interaction computing device, or both.

In some aspects, wherein initiating the action for the neurostimulation treatment further comprises providing a question to the patient related to the neurostimulation treatment, providing a question to the programmer related to the neurostimulation treatment, or both.

In some aspects, further comprising: receiving a response to the question from the patient, the question to the programmer, or both; and retraining the LLM based on the received response.

In some aspects, wherein the trained LLM further comprises a trained LLM that is trained at least on a data set that includes training on a plurality of past patient interactions that are local to the patient.

In some aspects, wherein querying the trained LLM generates both the programming recommendation and the recommended action, and wherein the recommended action further comprises providing a notification provided to the patient that is indicative at least of a scheduled time of application of the programming recommendation to the programmed neurostimulation device.

The above and other aspects, embodiments, features and benefits of the present disclosure will be readily apparent from the following detailed description.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neurostimulation system suitable for LLM-based neurostimulation programming recommendations;

FIG. 2 illustrates an embodiment of a stimulation device and a lead system, such as may be implemented in the neurostimulation system of FIG. 1;

FIG. 3 illustrates an embodiment of a programming device, such as may be implemented in the neurostimulation system of FIG. 1;

FIG. 4 illustrates an implantable neurostimulation system and portions of an environment in which the system may be used;

FIG. 5 illustrates an embodiment of an implantable stimulator and one or more leads of a neurostimulation system;

FIG. 6 illustrates an embodiment of a patient programming device for a neurostimulation system;

FIG. 7 illustrates an embodiment of data interactions among a clinician programmer device, a patient programming device, a program modeling system, and a data service for generating and implementing respective programs of defined parameter settings for operation of a neurostimulation device;

FIG. 8 illustrates an example diagram of a system for receiving inputs from patients;

FIG. 9 illustrates an example of a user interface configured to notify a patient of the application of an LLM-based neurostimulation programming recommendation;

FIG. 10 illustrates an embodiment of a data processing flow for affecting the neurostimulation treatment of a human patient, based on LLM-based neurostimulation programming recommendations;

FIG. 11 illustrates an example of a method for large language model (LLM)-based neurostimulation programming recommendations;

FIG. 12 illustrates another example of a method for LLM-based neurostimulation programming recommendations;

FIG. 13 illustrates a block diagram of an example computing system for LLM-based neurostimulation programming recommendations;

FIG. 14 illustrates a block diagram of an example of another computing system for LLM-based neurostimulation programming recommendations; and

FIG. 15 is a block diagram of an example machine including instructions that may be executed to cause the machine to perform any one of the methodologies herein.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

As noted, there is a need to improve neurostimulation programming recommendations for neurostimulation devices. That is, the systems, devices, and methods herein can utilize a trained LLM to generate programming recommendations for implantable electrical neurostimulation devices, for instance, in connection with the treatment of pain or related physiological conditions in a human subject (e.g., a patient). As an example, various systems and methods are described for querying a trained LLM with patient freeform text and similar forms of unstructured or unclassified data such as patient audio and/or patient video. The trained LLM can be trained to evaluate the current results or efficacy of neurostimulation treatment based on the patient input, and to make recommendations for changes or actions related to therapy objectives and desired outcomes (e.g., to improve efficacy of the neurostimulation treatment from a patient and/or programmer perspective). As a result, programming recommendations and/or recommended actions can be generated by the trained LLM, as detailed herein. For instance, the programming recommendations and/or recommended actions (e.g., questions, suggestions, etc.) can be provided to a programmer and/or a patient, as detailed herein. Hence, the systems, devices and methods herein are amenable to improving the efficacy of existing neurostimulation devices (e.g., those implanted in a patient), as detailed herein. Yet, in some embodiments, the systems, devices, and methods herein can also be utilized to screen or identifying pain or other symptoms in a perspective neurostimulation patient that may lend themselves to effective remediation via neurostimulation, as detailed herein. In either context, a trained LLM can be queried at least with patient input data to generate various information such as programming recommendations and/or recommended actions.

Accordingly, the systems, devices and methods herein involve the use of processing with machine-learning models such as a large language model (LLM). For purposes of this disclosure, a “large language model” is a computerized neural network of one million or more parameters, pre-trained on a data set of more than one million language tokens. One of ordinary skill will recognize a variety of LLM approaches and machine-learning techniques introduced in the art, including unidirectional and bidirectional frameworks, feed-forward and feedback connections, attention mechanisms, and transformers with a variety of features. Any of these tools are recognized as large language models within the art.

In various examples, the LLM can be trained to analyze various aspects associated with administration of neurostimulation treatment to a patient. For instance, the LLM can be trained to analyze programmer input data (clinician programmer device data, etc.) and/or patient input data (e.g., a patient programming device data which is indicative of real-time patient neurostimulation programming efficiency), etc. For instance, the LLM can be trained to analyze freeform text, audio, and/or images which are input from a patient undergoing neurostimulation treatment, as detailed herein. In some embodiments, the LLM can be trained on or otherwise analyze various types of programmer-patient interaction data such as voice to text data, text to voice, data, images of the patient, and/or voice to voice data obtained during a programmer-patient interaction (e.g., a communication session between a patient and a programmer). Similarly, in some embodiments the LLM can be trained on or otherwise analyze other forms for patient input data such as patient input data (audio, text, video, etc.) obtained during a communication session between the patient and a chatbot, etc. In some embodiments, the patient input data can be obtained directly from a patient (e.g., as audio, text, and/or video). Alternatively, or in addition, the patient input data can be obtained from another individual associated with the patient such as a caregiver, advocate/family member, clinician, among others. Hence, in such instances the patient input data is ‘derived from’ or ‘related to’ to support from others than the patient being treated.

In any case, the textual, audio, and/or visual LLM-based analysis of patient inputs can be used to produce corresponding scores or other indicators of an efficacy of the neurostimulation treatment. For instance, the scores can measure polarity or valence (i.e., the degree to which some outcome is “good” (positive valence) or “bad” (negative valence)). Scores produced from textual, audio, and/or visual LLM-based analysis of patient inputs may be used to then identify which device settings, programs, parameters, or operations are effective, ineffective, or problematic. Such information also may be used to generate programming recommendations and/or recommended actions, as detailed herein. Moreover, such information can be used to generate alerts, reports, or facilitate the retraining of a model (e.g., retraining of the trained LLM). In further examples, a LLM can be trained to translate the captured patient inputs to polarity (valence) scores. These polarity scores, which are paired with timestamps, then can be cross-referenced against device data (e.g., program usage, device on/off state, physiological state from a sensor, etc.) and/or another type of patient input (e.g., patient audio and/or images of the patient). The polarity of a particular type of input (e.g., text, audio and/or video) be directly determined as a result of sentiment analysis performed using any number of LLM-based processing techniques. The polarity of an input statement and the resulting patient state may be used, for instance, to identify the most effective settings of a neurostimulation program, directly from patient feedback collected over time.

In a further example, individual polarity scores may not necessarily be robust or fully indicative of a patient state. As a result, one or more filters may be applied to polarity values over time (including, in the simplest form, a moving average) and/or polarity scores from two or more different types of patient inputs (e.g., text input and video input such as images of the facial expressions of a patient) may be aggregated, so that the resultant polarity score is not improperly sensitive to individual patient inputs (e.g., text alone) and/or is not limited to text-based patient input. Additionally, it will be understood that some neurostimulation programming settings may have a longer effect or relevance (e.g., text comments provided by a patient may be relevant to the program used a number of hours ago, and not the current program e.g., as evidence by images of the patient currently smiling, etc.). A filtering mechanism may be used to take such changes and time-based effects into account.

In addition to use with aspects of programs and programming values, information from the evaluation of text, audio and/or video patient inputs may also be used to cause device actions (e.g., to run diagnostics on the neurostimulation device). In still other examples, information from the evaluation of textual, video, and/or audio patient inputs may also be used to provide informational content to a patient or to a clinician (e.g., to present guidance regarding the effects of treatment or ways to improve treatment outcomes), to provide a clinical triage system, or to update data records, among other effects.

In various embodiments, the present subject matter may be implemented using a combination of hardware and software designed to capture and analyze text (e.g., free form text), audio patient inputs, and/or other unstructured information from users, and related device data or context from a neurostimulation treatment. For instance, some examples are provided with reference to a mobile computing device (e.g., smartphone) app executing a user interface to collect freeform text, audio, and/or image inputs (e.g., facial expressions, etc.) that may be entered in response to questions displayed via the user interface or otherwise provided (e.g., providing the question as an audio output via the device). For instance, a computing system may use an application or chatbot (e.g., generating data for a smartphone app chat session or SMS message chat session) that presents questions or replies, in an effort to collect and process patient input provided in text (e.g., provided directly in freeform text from a patient response, provided from converted voice-to-text responses, or provided directly or indirectly with other interactions with various parties or entities) and/or that is provided as an audio response from the patient. Still other examples are provided with reference to a computing system platform which captures and evaluates data from sensors (e.g., wearable devices, implantable devices, or the neurostimulation device) that can be used to cross-reference or correlate freeform text, audio, image statements from a patient. Many of the following approaches are provided with specific reference to text, audio, image analysis and LLM-based processing, but it will be understood that such approaches may be supplemented or substituted with other technical implementations of text processing and data analysis involving artificial intelligence (AI), including models implementing machine learning, neural networks, decision trees, and the like.

It will be understood that a variety of the following embodiments may be operated to provide users such as patients, caregivers, clinicians, researchers, physicians, or others with the ability to monitor, collect and provide feedback, and adapt neurostimulation programs and neurostimulation effects (including, neurostimulation programming that provides a variation in the location, intensity, and type of defined waveforms and patterns in an effort to increase therapeutic efficacy and/or patient satisfaction). While neurostimulation therapies, such as SCS and DBS therapies, are specifically discussed as examples, the present subject matter may apply to other therapies that employ stimulation pulses of electrical or other forms of energy for treating chronic pain or physiological or psychological conditions.

The delivery of neurostimulation energy that is discussed herein may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc. Accordingly, the following drawings introduce the features of an example LLM-based neurostimulation system and how such LLM-based programming recommendations and/or LLM-based recommended actions (e.g., questions, suggestions, etc. that are provided to a patient and/or a programmer) may be employed to increase the efficacy of a neurostimulation system.

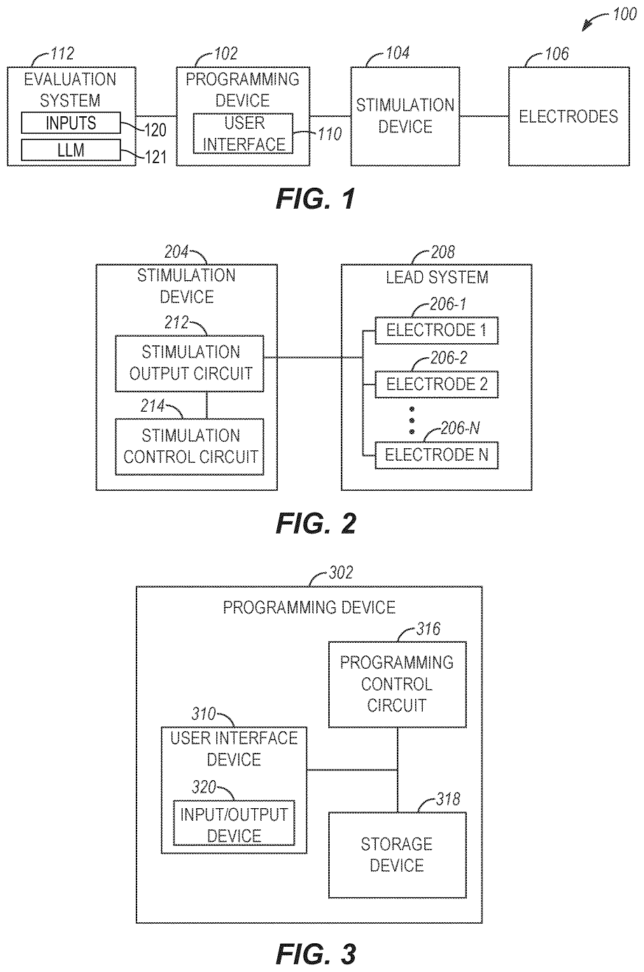

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and delivers neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are selected or programmable by a clinical user, such as a physician or other caregiver who treats the patient using system 100; however, some of the parameters may also be provided in connection with closed-loop programming logic and adjustment. Programming device 102 provides the user with accessibility to implement, change, or modify the programmable parameters e.g., in view of an LLM-based programming recommendation, as detailed herein. In various embodiments, programming device 102 is configured to be communicatively coupled to stimulation device 104 via a wired or wireless link.

In various embodiments, programming device 102 includes a user interface 110 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. These adjustments which may be included in the programming recommendations described herein may also include changing and editing values for the user-programmable parameters or sets of the user-programmable parameters individually (including values set in response to a therapy efficacy indication and/or an LLM-based programming recommendation). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The present approaches further provide examples of an LLM-based evaluation system 112, such as an LLM-based data analysis system, that is used to evaluate and improve the efficacy of neurostimulation treatment with stimulation device 104. This evaluation system 112 initiates an action related to the neurostimulation treatment based on inputs 120. The inputs may be manifested as text, audio, video/image inputs 120 such as those that may be directly collected from the patient and analyzed by the evaluation system 112, to then generate, via the LLM, a programming recommendation and/or a recommended action based on the inputs 120. For instance, the programming recommendation can include a recommendation to modify, start, stop, and/or monitor a neurostimulation treatment with stimulation device 104. Hence, the programming recommendation can include a recommendation to alter the neurostimulation treatment provided by the electrodes 106. For example, in some embodiments the recommendation can be a recommendation to modify therapy by decreasing therapy e.g., e.g., decreasing a frequency, amplitude, and/or quantity of electrodes utilized, etc., for instance, when it is determined that the decrease in therapy will not negatively impact a patient condition.

As described in more detail herein, a user, e.g., the patient, can provide inputs to the evaluation system 112, which are used to query a LLM 121 in the evaluation system 112. In some embodiments, inputs 120 can further include programmer inputs provided by a programmer or clinician (e.g., via a clinician interaction computing device or other programming device). The programmer inputs can include questions posed to a patient and/or actual inputs provided by the programmer to a clinician interaction computing device. For example, the programmer inputs can include a series of selections made regarding a particular neurostimulation program applied to a neurostimulation device (e.g., that is implanted in a patient or is associated with a patient undergoing a neurostimulation trial) and/or can include addition feedback (e.g., programmer notes such as those relating to an observed patient state from the perspective of the programmer, and/or programmer notes related to the series of selections made regarding a particular neurostimulation program applied to a neurostimulation device). That is, LLM 121 can be provided with the current neurostimulation device (e.g., a make/model/quantity of leads, etc.) providing neurostimulation therapy to a patient, a neurostimulation program currently applied to the neurostimulation device (e.g., a particular stimulation program and/or corresponding programming parameters, etc.). Hence, the LLM 121 can readily ascertain a status (e.g., a current status) of a neurostimulation device and can correlate the current status with any corresponding patient inputs/feedback regarding the same.

In some embodiments, the inputs 120 can be utilized to retrain the LLM 121 (e.g., to retrain the LLM 121 from a prior initial training of the LLM 121 such as an initial training based on subject matter expert or programmer inputs alone). The LLM 121 can be trained (and retrained) to analyze the inputs 120 to provide a programming recommendation and/or recommended actions to improve the efficiency of the neurostimulation treatment that is implemented by the stimulation device 104. This LLM-based evaluation using the LLM 121 may be based on a combination of natural language processing, sentiment analysis, rules, and other operational or treatment objectives that are identified. For instance, the LLM 121 can determine an appropriate action to take (e.g., an appropriate programming recommendation and/or recommended action) based on the state of the patient (e.g., as determined based on the inputs 120). Examples of patient states included but not limited to: a program or parameter change or recommendation to produce an improvement for a treatment objective (such as to address pain, increase mobility, reduce sleep disruption, and the like); diagnostic or remedial actions on the stimulation device 104; data logging or alerts to the patient or a clinician associated with the patient; and the like.

In some embodiments, the LLM 121 can, via one or more sensors in programming device and/or a patient interaction computing device, be configured to passively listen to (capture) patient audio and/or video (e.g., during a patient and programmer programming session, during a chatbot session, during normal use of the implantable neurostimulation device, and/or while a patient is making an e-diary entry etc.). In such embodiments, the patient interaction computing device can be configured with a selectable icon or button to permit the patient to selectively opt into or out of having audio and/or video passively captured by the device. In some embodiments, the passively captured audio and/or video (or a derivative thereof such as a text transcript of the audio) can be used to query the LLM 121.

In some embodiments, the LLM 121 can be configured (trained) to detect the presence of trigger phrases and/or key words in text or captured audio such as those associated with adequate or inadequate (e.g., worsening) neurostimulation therapy. For instance, trigger phrases can be employed that are hard-coded and either complete an entire action or trigger an action. For example, “my SCS On, Pain Attack!” (“My SCS On, Pain Attack” is an example of a specific trigger phrase e.g., that indicative of a need for providing a reprogramming request to the patient. In yet another example, the phrase “my SCS {fast} [Up/Down/On/Off]” (“my SCS” is the trigger phrase, where {} represents a rate of change e.g. fast slew rate or slow slew rate, and [] represents the direction/nature of change e.g. turn stim up, down, on, or off). In some embodiments, particular trigger phrases can correspond to particular actions such as particular reprogramming recommendations (e.g., to turn neurostimulation off and/or otherwise alter a neurostimulation parameter).

In some embodiments, the LLM 121 can be configured to detect the presence of inference phrases. As used herein, inference phrases refer to more generalized language (e.g., as compared to the trigger phrases) that can represent a patient experiencing adequate or inadequate (e.g., worsening) neurostimulation therapy. However, unlike trigger phrases, the inference phrases permit increased flexibility and/or accuracy of the LLM-based analysis of patient input. For instance, the patient statement “my pain is getting worse” includes the words “pain” + “getting” + “worse” which can be utilized to infer a patient condition regardless of a particular order and/or use of grammar associated with such words. Similarly, a patient statement such as “Ow” (or any 1 of multiple interjections or expletives) can result in the LLM 121 generating a programming recommendation to turn the neurostimulation device off and/or initiate performance of various recommended actions (e.g., turn a patient prompt on, at which point stimulation can be adjusted using verbal or manual commands from the patient). In some embodiments, the frequency over a time period and/or cadence of a word or phrase (e.g., which can be indicative of a patient state) can be utilized by the LLM 121 to generate a corresponding programming recommendation and/or recommended action.

In some embodiments, the LLM 121 may use a combination of trigger phrases and inference phrases when analyzing patient inputs such as text and/or audio inputs. For instance, as a safeguard trigger phrases and inference phrases may be timed to a short window (e.g. <5 s) (e.g., “pain getting worse” detected in 2 second vs. “pain”, “getting”, “worse” detected separately over 3 hours) and/or device may ask for patient confirmation responsive to an detected occurrence of a trigger phrase and/or inference phrase (e.g., may prompt the patient with various questions such as “are your currently experiencing more pain than usual”).

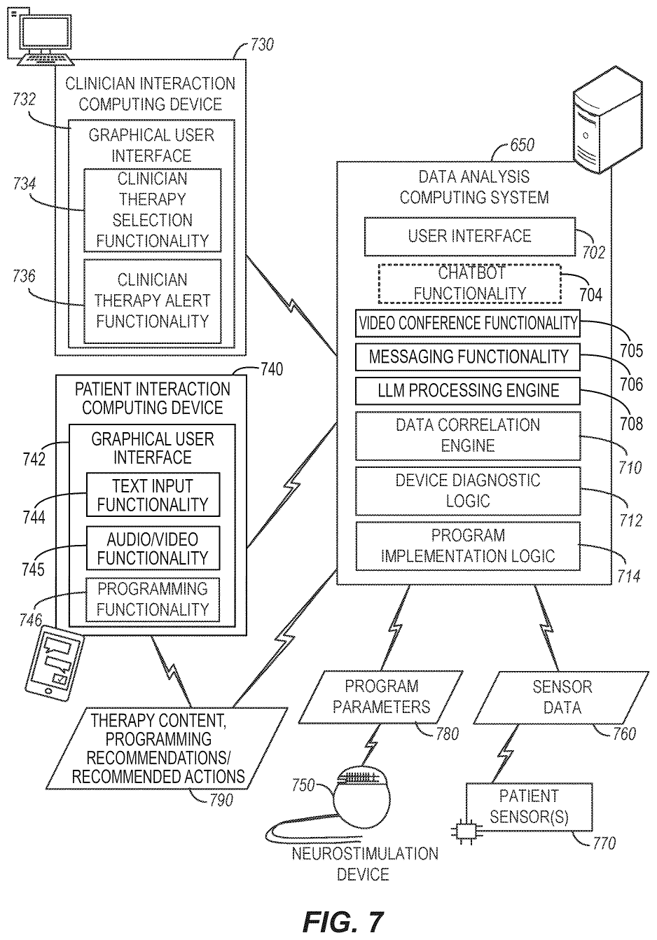

In some embodiments, visual patient inputs such as videos or still images of the patient (e.g., as taken by the patient interaction computing device 740 in FIG. 7) can be used to query the LLM 121. For instance, while a patient is making text based or other types of entries into an e-diary entry, facial expressions of the patient may be taken by on-board camera. In such instances, the facial expressions can be analyzed by the LLM 121 can be correlated, e.g., based on a time stamp or otherwise, with any other patient input e.g., such as the text or other types of entries made over the same time period to better access a patient state over the time period. The LLM 121 can be trained on various facial expressions and corresponding patient states related thereto (e.g., a smile indicates the patient is experiencing positive or acceptable neurostimulation therapy, etc.).

In any case, an LLM-based programming recommendation and/or a recommended action can be determined at least based on the patient inputs. For instance, a programming recommendation to change one or more parameters of a neurostimulation program can be generated by querying the LLM 121 at least with patient inputs. Example parameters that can be modified via a programming recommendation for a neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 6, a controller, e.g., controller 630 of FIG. 6, can implement program(s) and parameter setting(s) to affect a specific neurostimulation waveform, pattern, or energy output, using a program or setting in storage, e.g., external storage device 616 of FIG. 6, or using settings communicated via an external communication device 618 of FIG. 6 corresponding to the selected program. The implementation of such program(s) or setting(s) may further define a therapy strength and treatment type corresponding to a specific pulse group, or a specific group of pulse groups, based on the specific programs or settings. The LLM-based evaluation system 112 and the evaluation of the inputs 120 thereby provides a mechanism to determine the effectiveness of such programs or settings, and to identify issues and provide remediation for ineffective programs or settings, offer suggestions or recommendations for new programs or settings, or even to automatically change programs or settings.

Portions of the LLM-based evaluation system 112, the stimulation device 104 (e.g., implantable medical device), or the programming device 102 can be implemented using hardware, software, or any combination of hardware and software. Portions of the stimulation device 104 or the programming device 102 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 100 could also include a subcutaneous medical device (e.g., subcutaneous ICD, subcutaneous diagnostic device), wearable medical devices (e.g., patch-based sensing device), or other external medical devices.

FIG. 2 illustrates an embodiment of a stimulation device 204 and a lead system 208, such as may be implemented in neurostimulation system 100 of FIG. 1. Stimulation device 204 represents an embodiment of stimulation device 104 and includes a stimulation output circuit 212 and a stimulation control circuit 214. Stimulation output circuit 212 produces and delivers neurostimulation pulses, including the neurostimulation waveform and parameter settings implemented via a program selected or implemented with the user interface 110. Stimulation control circuit 214 controls the delivery of the neurostimulation pulses using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes. Those of ordinary skill in the art will understand that the neurostimulation system 100 may include additional components such as sensing circuitry for patient monitoring and/or feedback control of the therapy, telemetry circuitry, and power. The neurostimulation system 100 may also integrate with other sensors, or such other sensors may independently provide information for use with programming of the neurostimulation system 100 and/or training of the LLM 121.

The neurostimulation system 100 may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter” set. Each set of modulation parameters, including fractionalized current distribution to the electrodes (as percentage cathodic current, percentage anodic current, or off), may be stored and combined into a program that can then be used to modulate multiple regions within the patient.

The neurostimulation system may be configured to deliver different electrical fields to achieve a temporal summation of modulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields can be rotated or cycled through multiple times under a timing scheme, where each field is implemented using a timing channel. The electrical fields may be generated at a continuous pulse rate, or as bursts of pulses. Furthermore, an inter-pulse interval (i.e., the time between adjacent pulses), pulse amplitude, and pulse duration during the electrical field cycles may be uniform or may vary within the electrical field cycle. Some examples are configured to determine a modulation parameter set to create a field shape to provide a broad and uniform modulation field such as may be useful to prime targeted neural tissue with sub-perception modulation. Some examples are configured to determine a modulation parameter set to create a field shape to reduce or minimize modulation of non-targeted tissue (e.g., dorsal column tissue). Various examples disclosed herein are directed to shaping the modulation field to enhance modulation of some neural structures and diminish modulation at other neural structures. The modulation field may be shaped by using multiple independent current control (MICC) or multiple independent voltage control to guide the estimate of current fractionalization among multiple electrodes and estimate a total amplitude that provides a desired strength. For example, the modulation field may be shaped to enhance the modulation of dorsal horn neural tissue and to minimize the modulation of dorsal column tissue. A benefit of MICC is that MICC accounts for various in electrode-tissue coupling efficiency and perception threshold at each individual contact, so that “hotspot” stimulation is eliminated.

The number of electrodes available combined with the ability to generate a variety of complex electrical pulses, presents a huge selection of available modulation parameter sets to the clinician or patient. For example, if the neurostimulation system to be programmed has sixteen electrodes, millions of modulation parameter value combinations may be available for programming into the neurostimulation system. Furthermore, some SCS systems have as many as thirty-two electrodes, which exponentially increases the number of modulation parameter value combinations available for programming.

FIG. 3 illustrates an embodiment of a programming device 302, such as may be implemented in neurostimulation system 100. Programming device 302 represents an embodiment of programming device 102 and includes a storage device 318, a programming control circuit 316, and a user interface device 310. Programming control circuit 316 generates the plurality of stimulation parameters that control the delivery of the neurostimulation pulses according to the pattern of the neurostimulation pulses. The user interface device 310 represents an embodiment to implement the user interface 110.

In various embodiments, the user interface device 310 includes an input/output device 320 that is configured to receive user interaction and commands to load, modify, and implement neurostimulation programs and schedule delivery of the neurostimulation programs. In various embodiments, the input/output device 320 allows the user to create, establish, access, and implement respective parameter values of a neurostimulation program (e.g., in accordance with a LLM-based programming recommendation) through graphical selection (e.g., in a graphical user interface output with the input/output device 320), or other graphical input/output relating to therapy objectives, efficacy of applied treatment, user feedback, and the like. In various examples, the user interface device 310 can receive user input to initiate or control the implementation of the programs or program changes which are recommended, modified, selected, or loaded through use of an open or closed loop programming system, including those driven by LLM-based analysis as discussed herein.

In various embodiments, the input/output device 320 allows the patient or other user to apply, change, modify, or discontinue certain building blocks of a program and a frequency at which a selected program is delivered. In various embodiments, the input/output device 320 can allow the patient user to save, retrieve, and modify programs (and program settings) loaded from a clinical encounter, managed from the patient feedback computing device, or stored in storage device 318 as templates. In various embodiments, the input/output device 320 and accompanying software on the user interface device 310 allows newly created building blocks, program components, programs, and program modifications to be saved, stored, or otherwise persisted in storage device 318. Thus, it will be understood that the user interface device 310 may allow many forms of device operation and control, even if closed loop programming is occurring. The LLM-based analysis of inputs (e.g., inputs 120), discussed herein may be in addition to (or in place of) this user input and other forms of closed-loop or open-loop programming.

In one embodiment, the input/output device 320 includes a touchscreen. In various embodiments, the input/output device 320 includes any type of presentation device, such as interactive or non-interactive screens, and any type of user input device that allows the user to interact with a user interface to implement, remove, or schedule the programs. Thus, the input/output device 320 may include one or more of a touchscreen, keyboard, keypad, touchpad, trackball, joystick, and mouse. The logic of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

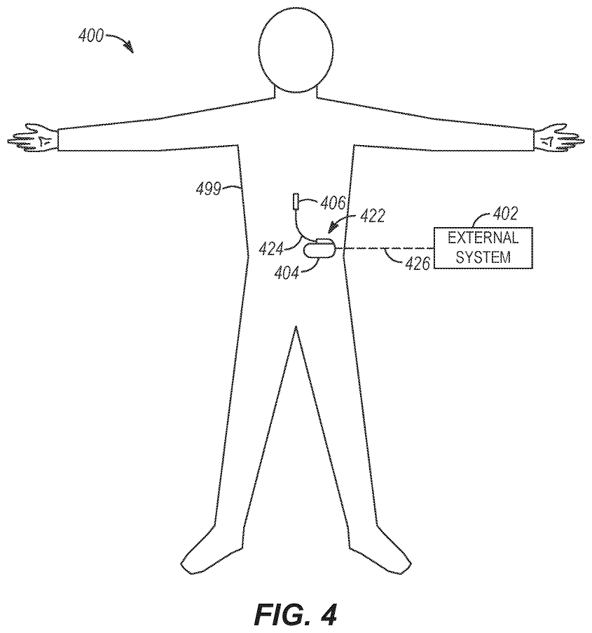

FIG. 4 illustrates an implantable neurostimulation system 400 and portions of an environment in which system 400 may be used. System 400 includes an implantable system 422, an external system 402, and a telemetry link 426 providing for wireless communication between an implantable system 422 and an external system 402. Implantable system 422 is illustrated in FIG. 4 as being implanted in the patient's body 499. However, in some embodiments the implantable system 422 can be located outside of the patient's body such as during an initial neurostimulation trial. The system is illustrated for implantation near the spinal cord. However, the neurostimulation system may be configured to modulate other neural targets.

Implantable system 422 includes an implantable stimulator 404 (also referred to as an implantable pulse generator, or IPG), a lead system 424, and electrodes 406, which represent an embodiment of the stimulation device 204, the lead system 208, and the electrodes 206, respectively. The external system 402 represents an embodiment of the programming device 302.

In various embodiments, the external system 402 includes one or more external (non-implantable) devices each allowing the user and/or the patient to communicate with the implantable system 422. In some embodiments, the external system 402 includes a programming device intended for the user to initialize and adjust settings for the implantable stimulator 404 and a remote-control device intended for use by the patient. For example, the remote-control device may allow the patient to turn the implantable stimulator 404 on and off and/or adjust certain patient-programmable parameters of the plurality of stimulation parameters e.g., in accordance with a programming recommendation generated by a LLM, as described herein. The remote-control device may also provide a mechanism to receive and process feedback on the operation of the implantable neurostimulation system. Feedback may include metrics or an efficacy indication reflecting perceived pain, effectiveness of therapies, or other aspects of patient comfort or condition. Such feedback may be automatically detected from a patient's physiological state, collected from other sensors or devices (not shown), or manually obtained from user input entered in a user interface (such as with the user input scenarios discussed herein). Such feedback and other information may comprise the device data evaluated as part of association and matching with inputs such as freeform text, audio, video inputs from a patient. Such feedback can be obtained from patient input data, programmer input data, or both.

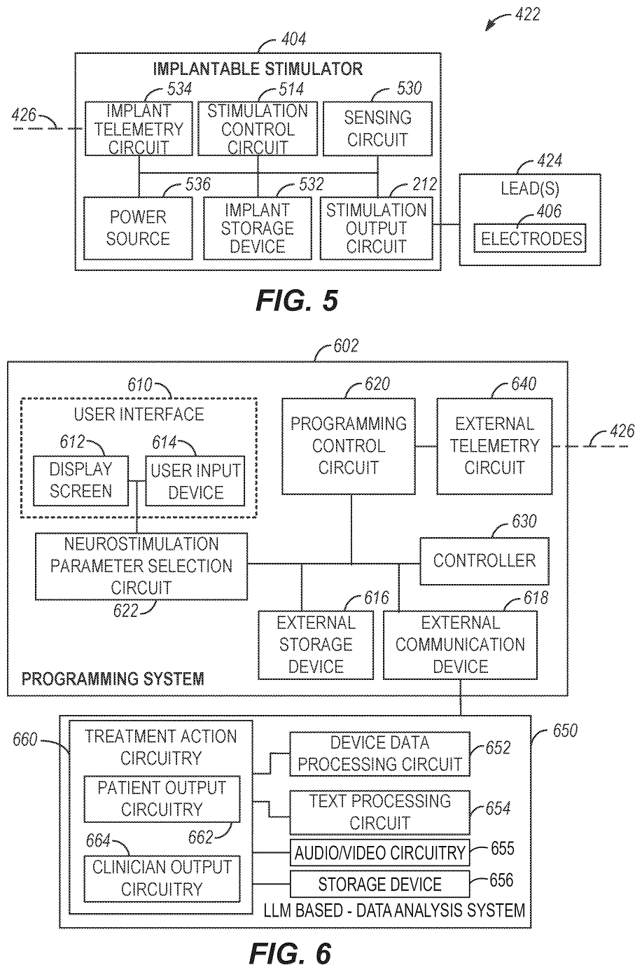

FIG. 5 illustrates an embodiment of the implantable stimulator 404 and the one or more leads 424 of an implantable neurostimulation system, such as the implantable system 422. The implantable stimulator 404 may include a sensing circuit 530 used for an optional sensing capability, stimulation output circuit 212, a stimulation control circuit 514, an implant storage device 532, an implant telemetry circuit 534, and a power source 536. The sensing circuit 530, when included and needed, senses one or more physiological signals for purposes of patient monitoring and/or feedback control of the neurostimulation, including in the closed loop programming processes discussed herein. Examples of the one or more physiological signals include neural and other signals indicative of a condition of the patient that is treated by the neurostimulation and/or a response of the patient to the delivery of the neurostimulation.

The stimulation output circuit 212 is electrically connected to electrodes 406 through the one or more leads 424, and delivers each of the neurostimulation pulses through a set of electrodes selected from the electrodes 406. The stimulation output circuit 212 can implement, for example, the generating and delivery of a customized neurostimulation waveform (e.g., implemented from a parameter of a program selected with the closed-loop programming system) to an anatomical target of a patient.

The stimulation control circuit 514 represents an embodiment of the stimulation control circuit 214 and controls the delivery of the neurostimulation pulses using the plurality of stimulation parameters specifying the pattern of the neurostimulation pulses. In one embodiment, the stimulation control circuit 514 controls the delivery of the neurostimulation pulses using the one or more sensed physiological signals and processed input from patient feedback interfaces. The implant telemetry circuit 534 provides the implantable stimulator 404 with wireless communication with another device such as a device of the external system 402, including receiving values of the plurality of stimulation parameters from the external system 402. The implant storage device 532 stores values of the plurality of stimulation parameters, including parameters from one or more programs which are activated, de-activated, or modified using the approaches discussed herein.

The power source 536 provides the implantable stimulator 404 with energy for its operation. In one embodiment, the power source 536 includes a battery. In one embodiment, the power source 536 includes a rechargeable battery and a battery charging circuit for charging the rechargeable battery. The implant telemetry circuit 534 may also function as a power receiver that receives power transmitted from external system 402 through an inductive couple.

In various embodiments, the sensing circuit 530, the stimulation output circuit 212, the stimulation control circuit 514, the implant telemetry circuit 534, the implant storage device 532, and the power source 536 are encapsulated in a hermetically sealed implantable housing. In various embodiments, the lead(s) 424 are implanted such that the electrodes 406 are placed on and/or around one or more targets to which the neurostimulation pulses are to be delivered, while the implantable stimulator 404 is subcutaneously implanted and connected to the lead(s) 424 at the time of implantation.

FIG. 6 illustrates an embodiment of a programming system 602 used as part of an implantable neurostimulation system, such as the external system 402, with the programming system 602 configured to send and receive device data (e.g., commands, parameters, program selections, information). FIG. 6 also illustrates an embodiment of an LLM-based data analysis computing system (e.g., data analysis system) 650, communicatively coupled to the programming system 602, with the data analysis computing system 650 used to perform data analysis on inputs (e.g., inputs 120 such as freeform text, audio, video/image capture from a patient) and device data in connection with neurostimulation treatment by the implantable neurostimulation system.

The programming system 602 represents an embodiment of the programming device 302, and includes an external telemetry circuit 640, an external storage device 616, a programming control circuit 620, a user interface device 610, a controller 630, and an external communication device 618, to effect programming of a connected neurostimulation device. The operation of the neurostimulation parameter selection circuit 622 enables selection, modification, and implementation of a particular set of parameters or settings for neurostimulation programming. The particular set of parameters or settings that are selected, modified, or implemented may be based on LLM-based analysis of inputs, such as discussed with reference to the text, audio, and/or image/video analysis as described herein.

The external telemetry circuit 640 provides the closed loop programming system 602 with wireless communication to and from another controllable device such as the implantable stimulator 404 via the telemetry link 426, including transmitting one or a plurality of stimulation parameters (including selected, identified, or modified stimulation parameters of a selected program) to the implantable stimulator 404. In one embodiment, the external telemetry circuit 640 also transmits power to the implantable stimulator 404 through inductive coupling.

The external communication device 618 may provide a mechanism to conduct communications with a programming information source, such as a data service, program modeling system, to receive program information, settings and values, models, functionality controls, or the like, via an external communication link (not shown). In a specific example, the external communication device 618 communicates with the data analysis computing system 650 to obtain commands or instructions in connection with parameters or settings that are selected, modified, or implemented based on LLM-based analysis from the data analysis computing system 650. The external communication device 618 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 640 and the external communication device 618 are depicted as separate components within the closed-loop programming system 602, the functionality of both of these components may be integrated into a single communication chipset, circuitry, or device.

The external storage device 616 stores a plurality of existing neurostimulation waveforms, including definable waveforms for use as a portion of the pattern of the neurostimulation pulses, settings and setting values, other portions of a program, and related treatment efficacy indication values. In various embodiments, each waveform of the plurality of individually definable waveforms includes one or more pulses of the neurostimulation pulses and may include one or more other waveforms of the plurality of individually definable waveforms. Examples of such waveforms include pulses, pulse blocks, pulse trains, and train groupings, and programs. The existing waveforms stored in the external storage device 616 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 616 may also store a plurality of individually definable fields that may be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program may be correlated to the control of these waveforms and definable fields.

The programming control circuit 620 represents an embodiment of a programming control circuit 316 and may translate or generate the specific stimulation parameters or changes which are to be transmitted to the implantable stimulator 404, based on the results of the neurostimulation parameter selection circuit 622. The pattern may be defined using one or more waveforms selected from the plurality of individually definable waveforms (e.g., defined by a program) stored in an external storage device 616. In various embodiments, the programming control circuit 620 checks values of the plurality of stimulation parameters against safety rules to limit these values within constraints of the safety rules. In one embodiment, the safety rules are heuristic rules.

The user interface device 610 represents an embodiment of the user interface device 310 and allows the user (including a patient or clinician) to provide input relevant to therapy objectives, such as to switch programs or change operational use of the programs, for instance, responsive to LLM-based programming recommendations that are provided e.g., displayed via the user interface device 610. The user interface device 610 includes a display screen 612, a user input device 614, and may implement or couple to the parameter selection circuit 622, or data provided from the data analysis computing system 650. The display screen 612 may include any type of interactive or non-interactive screens, and the user input device 614 may include any type of user input devices that support the various functions discussed in this document, such as a touchscreen, keyboard, keypad, touchpad, trackball, joystick, and mouse. The user interface device 610 may also allow the user to perform other functions where user interface input is suitable (e.g., to select, modify, enable, disable, activate, schedule, or otherwise define a program, sets of programs, provide feedback or input values, or perform other monitoring and programming tasks). Although not shown, the user interface device 610 may also generate a visualization of such characteristics of device implementation or programming, and receive and implement commands to implement or revert the program and the neurostimulator operational values (including a status of implementation for such operational values). These commands and visualization may be performed in a review and guidance mode, status mode, or in a real-time programming mode.

The controller 630 can be a microprocessor that communicates with the external telemetry circuit 640, the external communication device 618, the external storage device 616, the programming control circuit 620, the parameter selection circuit, and the user interface device 610, via a bidirectional data bus. The controller 630 can be implemented by other types of logic circuitry (e.g., discrete components or programmable logic arrays) using a state machine type of design. As used in this disclosure, the term “circuitry” should be taken to refer to discrete logic circuitry, firmware, or to the programming of a microprocessor.

The data analysis computing system 650 is configured to operate treatment action circuitry 660, which may produce or initiate certain actions on the basis of device data (received and processed by device data processing circuit 652) and patient inputs (e.g., received and processed by text processing circuit 654 and/or the audio/video processing circuitry 655). The treatment action circuitry 660 may identify one or more actions (e.g. programming recommendations and/or recommended actions such as posing one or more questions pertaining to the patient and/or programmer) related to the neurostimulation treatment, and provide outputs to a patient or a clinician using patient output circuitry 662 or clinician output circuitry 664 respectively. Such outputs and actions provided by the outputs are based on the evaluation and detection of particular patient states and device states from LLM-based analysis of patient inputs (e.g., freeform text, audio, image) and associated device data, discussed in more detail below.

The data analysis computing system 650 also is depicted as including a storage device 656 to store or persist data related to the device data, patient inputs, patient or clinician output, and related settings, logic, or algorithms. Other hardware features of the data analysis computing system 650 are not depicted for simplicity, but are suggested from functional capabilities and operations in the following figures.

As will be understood, patients who are experiencing chronic pain are often willing to provide detailed information regarding their current medical state responsive to questions. Freeform text in the form of a narrative, explanatory statement, or interjection is easy for patients to produce, and can provide many details regarding a patient's actions, physiological and physiological state, prior historical events, and can reflect both objective and subjective results of neurostimulation treatment. Video and/or audio (e.g., during a video call and/or in-person communication session between a patient and a programmer) and/or freeform text (e.g., entered via a patient during a chat session with a chatbot), however, can be time-consuming or difficult for physicians and clinicians to interpret, especially when patient feedback may be contradictory, ambiguous, and/or is incomplete without additional context (e.g., images indicate that the patient is visually wincing in pain). Capturing patient feedback an analyzing the patient input/feedback with the present LLM-based systems may provide many new data points for treatment outcomes, and provide a basis for determining whether or why a particular neurostimulation treatment (and treatment program, programming value, programming effect) is or is not effective and provide a recommended action (e.g., a recommended programming change and/or additional questions to facilitate determination of a recommended programming change) automatically.

Prior approaches for obtaining feedback from neurostimulation have often attempted to collect subjective data from a patient regarding specific aspects of pain or treatment. Often, prior approaches would use constrained inputs such as visual or numerical scales of pain or discomfort, multiple choice questions and answers, or structured inputs to obtain information from a patient. These inputs often fail to capture the nuance and the significance of historical events, and do not capture the surrounding context that is occurring from a patient. In contrast, the following approaches provide a system which can efficiently and quickly interpret patient input via an LLM, determine a patient state based on the interpreted patient inputs, and produce useful outcomes for diagnosis, treatment, and remediation relevant to neurostimulation device operation (e.g., provide LLM-based programming recommendations to improve the efficacy of the neurostimulation treatment for the particular patient providing the real-time inputs).

FIG. 7 illustrates, by way of example, an embodiment of data interactions among the data analysis computing system 650 and clinician and patient interaction computing devices 730, 740, for operation of a neurostimulation device 750 based on patient inputs (e.g., LLM-based analysis of audio and/or freeform text input). The data analysis computing system 650 identifies operations related to the neurostimulation treatment based on the analysis of input text, such as diagnostic actions, alerts, content or programming recommendations, or programming actions. Such programming actions (and operational actions based on programming recommendations) may be implemented on the neurostimulation device 750 (e.g., using the programming techniques discussed above). The data analysis computing system 650 identifies and initiates these actions through the execution of one or more data analysis engines, such as an LLM processing engine 708 which is trained to determine programming recommendations and/or recommended actions that facilitate subsequent programming recommendations based on responses thereto, and data correlation engine 710 which determines a state of treatment from historical or current operation of the neurostimulation device 750. In some examples, the determined state of treatment may be based on correlating the historical use of a neurostimulation program or set of parameters with the current state of a patient e.g., as determined based on the LLM analysis via the LLM processing engine 708 (e.g., identifying that a pain condition became worse after beginning use of a particular program at a previous point in time).

Specifically, the LLM-based data analysis computing system 650 operates the LLM processing engine 708 to analyze input originating from a human patient that is relevant to neurostimulation treatment. The analysis of such input may include using one or more forms of text parsing, linguistic analysis, among other types of inputs such as visual input (e.g., video and/or image inputs) and/or audio inputs. The patient inputs may be received via a user interface 702 of the LLM-based data analysis computing system 650, such as provided from chatbot functionality 704, from video-conferencing functionality 705, or messaging functionality 706. The patient inputs also may be provided from a patient interaction computing device 740, or other third-party devices and platforms not depicted.

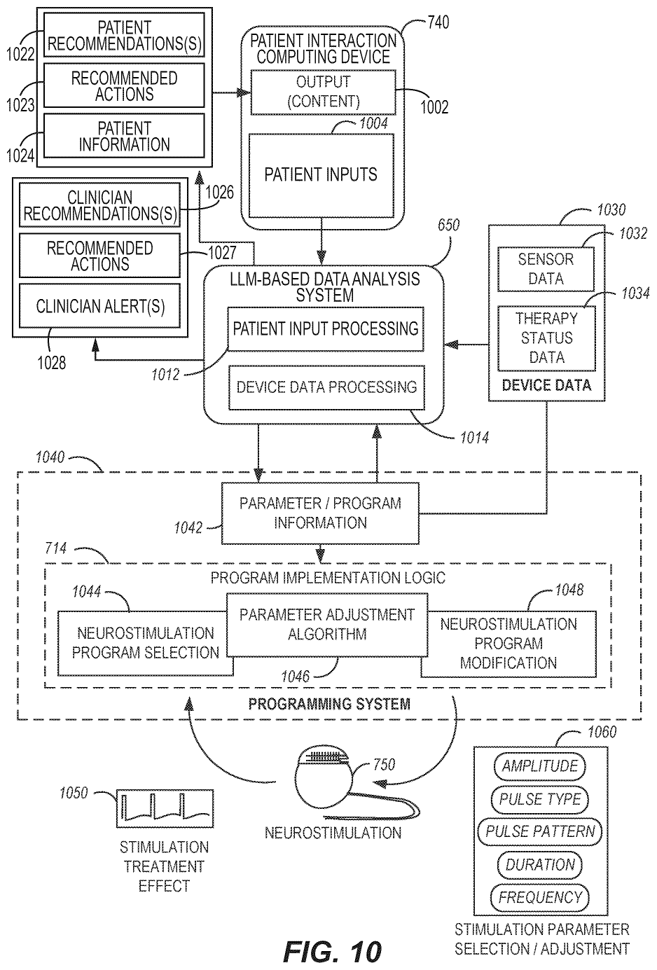

The LLM-based data analysis computing system 650 also operates data correlation engine 710 to correlate (e.g., identify, match, associate) device state data and patient state data, device diagnostic logic 712 to evaluate operational or conditions from the neurostimulation device 750, and program implementation logic 714 to effect changes in programming to the neurostimulation device 750. In an example, the program implementation logic 714 enables control, modification, selection, or specification of neurostimulation programming parameters, in an automatic, suggested, or manual fashion. Additional details regarding the programming of the device 750 is provided with reference to FIG. 10.

In an example, the LLM processing engine 708 applies one or more approaches for analysis of text, images, and/or audio received from the patient. Thus, unlike previous approaches such as those that are limited to text recognition and/or text analysis, the LLM processing engine 708 can analyze additional types of patient data (e.g., video and/or audio) to provide a more accurate indication of a state of a patient (e.g., to more accurately ascertain a degree of pain a patient is experiencing) and/or in some instances can account to environmental factors that may be relevant for determination of a patient condition. For instance, the LLM processing engine may be configured to determine a time of day, weather, a patient location (e.g., home, work), and/or various other environmental factors that may affect or be perceived by the patient to affect a patient state. Alternately, or in addition, the LLM processing engine 708 can be configured to derive an aggregate patient condition based on two or more types of patient input (e.g., from both audio and video images received during a video conferencing session between a patient and a programmer). Thus, at least due to the determination of environmental factors and/or determination of an aggregated patient state based on two or more different types of patient inputs (e.g., at least two of patient audio, patient video, and patient text), the approaches herein can yield more accurate determination of a patient state.

The LLM processing engine 708 represents at least an LLM stored in one or more systems which may be local to or remote from computing devices and systems described herein. The LLM may be customized for this purpose or may be a general-purpose language model. The LLM may be cloud-based, remotely run, or locally instantiated. In some cases, the LLM may be run as part of a computer device such as the LLM-based data analysis computing system 650. The LLM processing engine 708 may in some cases include further filtration or processing associated with LLM input and/or input, such as tokenizing queries, formatting results, and/or recording exchanged data for later use and improvement. In some embodiments, the methods herein can be employed using a cloud-based system with at least some local processing capabilities being present on a clinician programmer device.

In an example, the patient interaction computing device 740 is a computing device (e.g., personal computer, tablet, smartphone) or other form of user-interactive device which receives and provides interaction with a patient using a graphical user interface 742, text input functionality 744, audio/video input functionality 745, and programming functionality 746. For instance, the text input functionality 744 may receive freeform text from a patient via questionnaires, surveys, messages, or other textual inputs. Such inputs may provide text related to pain or satisfaction, that can be used to identify a psychological or physiological state of the patient, neurostimulation treatment results, or related conditions. The audio/video input functionality 745 may receive other forms of non-text input functionality such as patient audio captured via a microphone of the patient interaction computing device 740 and/or patient video capture via a camera of the patient interaction computing device.

The patient interaction computing device 740 is also depicted as including the programming functionality 746, to provide one or more outputs in the graphical user interface related to programming control or implementation. The programming functionality 746 specifically may provide the patient with therapy content and programming recommendations 790 generated by the LLM-based data analysis computing system 650. Other form factors and interfaces such as audio interfaces and text interfaces may also be substituted for or augmented with the graphical user interface 742.

The clinician interaction computing device 730 may include a graphical user interface 732, which implements clinician therapy selection functionality 734 and clinician therapy alert functionality 736, offering similar capabilities to the graphical user interface 742 for the patient, but adapted for use by a clinician (e.g., to provide enhanced functionality or features for physician control). Although not depicted, the therapy content, recommended actions, and/or programming recommendations 790 can also be presented via the graphical user interface 732 and/or another user interface.

In an example, the LLM-based data analysis computing system 650 generates, selects, or communicates therapy content, programming recommendations and/or recommended actions 790 to the patient interaction computing device 740 or clinician interaction computing device 730. Such content including the recommendations 790 and/or recommend actions 791 are provided based on aspects of a correlated patient and device state, from a patient state detected from the LLM-based processing of patient inputs. The therapy content and programming recommendations 790 may include a recommendation or identification of the type of therapies to apply, instructions, recommendations, or feedback (including clinician recommendations, behavioral modifications, etc., selected for the patient). The therapy content and recommendations 790 also may provide relevant information based on the sensor data 760 or other neurostimulation state monitoring performed on the patient.

The LLM-based data analysis computing system 650 may utilize sensor data 760 from one or more patient sensors 770 (e.g., wearables, sleep trackers, motion tracker, implantable devices, etc.) among one or more internal or external devices. The sensor data 760 may be used in addition to the program parameters 780, to determine a customized and current state of the patient condition or neurostimulation treatment results. In various examples, the neurostimulation device 750 includes sensors which contribute to the sensor data 760 evaluated by the LLM-based data analysis computing system 650.

In an example, the patient sensors 770 are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized. In any case, the sensor data obtained from the patient sensors 770 can, in some embodiments, be utilized to query the LLMs herein (e.g., in addition to other patient inputs such as text, audio, and video input) to obtain LLM-based programming recommendations and/or recommended actions. Hence, unlike other approaches such as those that are reliant solely on text recognition and/or analysis (e.g., are limited to text analysis via non-LLM based approaches such as natural-language model processing), the approaches herein can automatically include additional patient inputs (e.g., obtained from patient sensors) into LLM-based programming recommendations and/or recommended programming actions. Thus, the approaches herein can more accurately assess a patient's condition and thereby may more effectively tailor neurostimulation treatment to the particular patient (e.g., generate a programming recommendation to improve the patient's condition relative to an initial or prior patient condition).

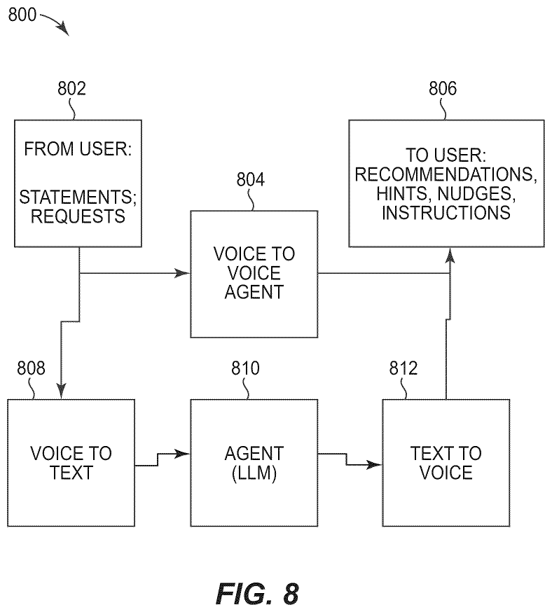

FIG. 8 illustrates an example diagram of a system 800 for receiving inputs from patients. The system can be deployed at least in part on one or more of the computing devices described herein such as a clinician interaction computing device. Various types of user interfaces and interactions which directly or indirectly receive various inputs such as voice and text inputs from a human patient and/or from a programmer. For example, at 802 a statement or request from a user (e.g., a patient, caretaker, programmer, clinician, medical rep, or other individual) can be received. In some instances, the statement or request can be manifested as an audio input. In such instances, the audio input can be provided, via a voice-to-voice agent 804, to another user, as indicated at 806. This pathway via the voice-to-voice agent represents a traditional pathway employed during an audio or video call, for instance, between a patient and programmer. However, in various embodiments described herein, the statement or request received at 802 can be conveyed to a trained LLM to train (e.g., retrain) and/or query the trained LLM with the statement or request (or a derivative thereof) received at 802. For example, the statement or request received at 802 can be provided to a voice-to-text agent at 808. The voice to text agent can be configured to convert the voice input to a text input. The resultant text generated at 808, which is a derivative of the initial statement or request at 802, can be provided to the LLM, again to train and/or query the trained LLM 121 which is a LLM such as those described herein (e.g., that is trained to generate programming recommendations, etc.). For instance, the resultant text may be indicative of a patient state and can be used to query the LLM 121 to determine a programming recommendation for the patient, among other possibilities. At 812, the text provided to the LLM and/or a text-based result of querying the LLM 121 can be converted, by the text to voice agent, to audio (e.g., voice). The audio or voice content can then be provided to the user, at 806.

Hence, such text content may include the results from voice-to-text converted from voice phone or online calls with a medical device representative or a patient care entity. Further, it will be understood that relevant text data may be provided from voice, text, or multi-modal input from multiple channels (e.g., SMS text messages, an email, an app, a website, a chatbot, a virtual universe meeting, etc.). Moreover, such text data may be provided from the conversion of voice-to-text from in-app voice recordings, voice chats, voicemails, or voice interactions with virtual assistants or agents (e.g., Amazon® Alexa, Google® Assistant, Apple® Siri, etc.). Analysis may also be performed on voice recordings directly to obtain relevant characteristics, such as to identify the vocal tone of the statement (e.g., analyzing the auditory signal itself to identify physiological or psychological characteristics of the human patient such as calmness, irritation, sadness, etc.). It will be understood that the collection of patient input may extend into a variety of settings, being integrated into multiple products/apps, including feedback captured during patients' normal activities, clinician visits, and other events, thus capturing a more realistic view of a patient state (e.g., based on a plurality of different types of patient input and/or based on patient input gathered in a plurality of different contexts or environments).

In some embodiments, patients can be triaged based on the LLM-based analysis of patient inputs. Such triaging performed based on LLM-based patient input analysis can help make it easier for clinicians (including device manufacturer representatives, physicians, etc.) to identify which patients are in need of what type of support and/or identify a priority for addressing the patients in need of support. For example, when the LLM such as the LLM 121 determines that a neurostimulation treatment is inadequate (e.g., does not satisfactorily cease pain) based on patient inputs, the LLM 121 or a data analysis computing system such as those described herein can cause initiation of one or more actions. For instance, continuing with the above example, a first action can be initiated to provide a clinician alert (e.g., to a clinician interaction computing device) that the pain treatment is not effective, and a second action can be initiated to provide patient alert with a programming recommendation such as a recommendation to implement a new program setting or alter a neurostimulation program.

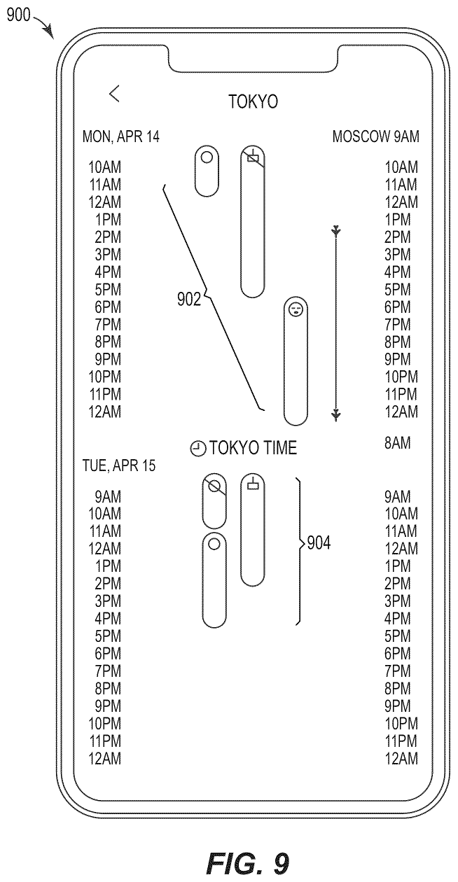

FIG. 9 is an example of a user interface 900 in a patient interaction computing device (e.g., patient interaction computing device 740 as illustrated in FIG. 7). As illustrated at 902, the user interface 900 can include indicators associated with a current neurostimulation program and/or programming parameter. For instance, the indicators can include a current day (e.g., Monday)/time and/or corresponding indicators for the current neurostimulation program and/or programming parameter displayed concurrently therewith as illustrated in FIG. 9.

In some embodiments, the user interface can be configured to provide a notification to the patient that is indicative of a scheduled time of application of a programming recommendation (e.g., an LLM-based programming recommendation) to the programmed neurostimulation device that is implanted in a patient. For instance, as illustrated in FIG. 9, a future day (e.g., Tuesday)/time and/or corresponding indicators for the programming recommendation and/or a programming parameter included in the programming recommendation can be displayed concurrently therewith as illustrated at 904, in FIG. 9. Hence, the approaches herein can provide LLM-based programming recommendations and can provide a patient advance notification of any application of the LLM-based programming recommendation to the implanted neurostimulation device. Such notification can improve a patient's experience and/or provide an additional avenue for fielding patient input (e.g., subsequent to providing the patient notification but prior to application of the programming recommendation, etc.)

FIG. 10 illustrates, by way of example, an embodiment of a data processing flow affecting the neurostimulation treatment of a human patient, based on implemented LLM-based patient input processing 1012 and device data processing 1014 functions. Here, additional details are provided on the data flow between the LLM-based data analysis computing system 650, an example user interface (graphical user interface 742). Other user interfaces and actions are not depicted for simplicity.

In this example, patient input 1004 (e.g., freeform text, audio/video inputs) is obtained by the LLM-based data analysis computing system 650 from the patient interaction computing device 740. FIG. 10 also depicts the evaluation of device data 1030, such as sensor data 1032, therapy status data 1034, and other treatment aspects which may be obtained or derived from the neurostimulation device or related neurostimulation programming. Also, in this example, output 1002 (e.g., content) is obtained from the LLM-based data analysis computing system 650 such as in the form of patient recommendations 1022, recommended actions 1023, or patient information 1024. The LLM-based data analysis computing system 650 may separately provide clinician recommendations 1026, recommended actions 1027, clinician alerts 1028, or other related actions.

The remainder of the data processing flow illustrates how data processing results from the LLM-based data analysis computing system 650 can be used to effect programming, such as in a closed loop (or partially-closed-loop) system. A programming system 1040 uses parameter or program information 1042 provided from the LLM-based data analysis computing system 650 as an input to program implementation logic 714. The program implementation logic 714 may be implemented by a parameter adjustment algorithm 1046, which affects a neurostimulation program selection 1044 or a neurostimulation program modification 1048. For instance, some parameter changes may be implemented by a simple modification to a program operation; other parameter changes may require a new program to be deployed. The results of the parameter or program changes or selection results in definition or adjustment to various stimulation parameters 1060 at the neurostimulation device 750, causing a different or new stimulation treatment effect 1050.

By way of example, operational parameters of the neurostimulation device which are generated, identified, or evaluated by the present systems and techniques may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

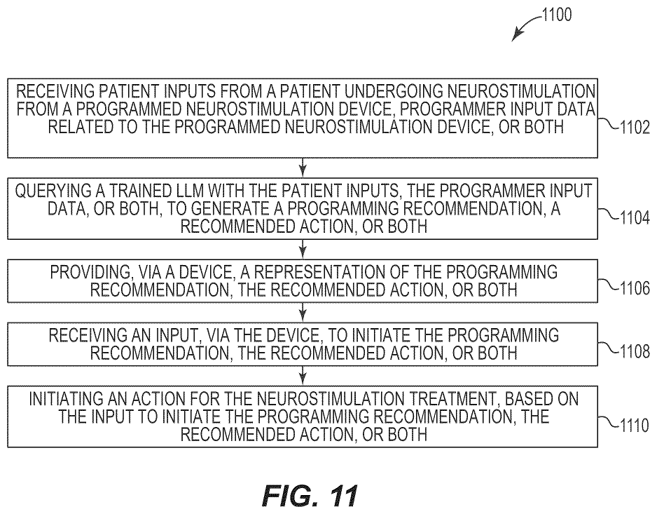

FIG. 11 illustrates an example method 1100 for large language model (LLM)-based neurostimulation programming recommendations. The method 1100 can be performed by one or more of the devices and systems herein. For instance, the method 1100 can be performed by one or more of the devices in the system 100.

At 1102, the method 1100 receiving patient inputs from a patient undergoing neurostimulation from a programmed neurostimulation device, programmer input data related to the programmed neurostimulation device, or both, as described herein. For instance, a selection may be made via a graphical user interface of other input mechanism (e.g., keyboard, mouse, etc.) on a patient interaction computing device (e.g., patient interaction computing device 740 in FIG. 7) and/or a clinician interaction computing device (clinician interaction computing device 730), among other possibilities. In some embodiments, the received patient inputs can include patient feedback on a currently applied (or previously applied) neurostimulation program to a neurostimulation device (e.g., that is implanted in and providing neurostimulation therapy to the patient). Similarly, in some embodiments, the received programmer input data can include programmer inputs on a currently applied (or previously applied) neurostimulation program to a neurostimulation device.

As mentioned, the patient inputs can be manifested as text, audio, and/or images (e.g., video) of the patient such as text, audio, and/or images gathered via a patient interaction computing device (e.g., patient interaction computing device 740 in FIG. 7). In some embodiments, the patient inputs can include at least text, at least audio, and/or at least images. For instance, in some embodiments, the patient inputs can be manifested as two or more of text (e.g., free text obtained via a programmer-patient chat session and/or via a patient-chatbot chat session), audio (e.g., obtained passively at the same time as the text is obtained), and/or images (e.g.,) video obtained passively at during the same time as the text, audio, image is obtained). Hence, unlike other approaches, the in some embodiments an aggregated patient input including at least two of the patient text, audio, and/or images can be employed to more accurately ascertain a current patient status (e.g., the effectiveness of a current neurostimulation treatment for the patient). The enhanced accuracy of a patient's status can in turn lead to improved LLM-based programming recommendations and/or recommended actions (e.g., questions to ask the patient), as described herein.

For instance, at 1104, the method 1100 includes querying a trained LLM with the patient inputs, the programmer input data, or both, to generate a programming recommendation, a recommended action, or both, as described herein. At 1106, the method 1100 can include providing a representation of a programming recommendation such as displaying, via a display of a device, a representation of the programming recommendation, the recommended action, or both, as described herein. In such instances, the representation can be provided to a clinician interaction computing device, a patient interaction computing device, or both, as described herein.

In some embodiments, the method 1100 includes generating, via the trained LLM, a programming recommendation. In some embodiments, the programming recommendation can be based on predefined neurostimulation parameters in the trained LLM. Examples of predefined neurostimulation parameters include available programming settings for the programmed neurostimulation device, clinician approved programming settings for the programmed neurostimulation device, previously utilized programming settings for the programmed neurostimulation device, and/or any combination thereof. In some embodiments, the predefined neurostimulation parameters can be specified by a neuromodulation subject matter expert input. Stated differently, in some embodiments, a neuromodulation subject matter expert input can train (e.g., initially train) the LLM with available programming settings for the programmed neurostimulation device, clinician approved programming settings for the programmed neurostimulation device, previously utilized programming settings for the programmed neurostimulation device, and/or any combination thereof, among other possibilities.

At 1108, the method 1100 includes receiving an input, via the device, to initiate the programming recommendation, the recommended action, or both. For instance, the programming recommendation (e.g., to adjust a setting or programming parameter in a currently applied neurostimulation program or apply a different neurostimulation program to the neurostimulation device than a currently applied neurostimulation program to the neurostimulation device) can be provided to a patient interaction computing device and the patient can accept the programming recommendation (e.g., via an input to the patient interaction computing device).

At 1110, the method 1100 can include initiating an action for the neurostimulation treatment, based on the input to initiate the programming recommendation, the recommended action, or both. The action can be altering a neurostimulation treatment applied to a patient e.g., implementing a programming recommendation and/or can include other types of actions such as prompting the patient and/or a programming with one or more questions related to the neurostimulation treatment.

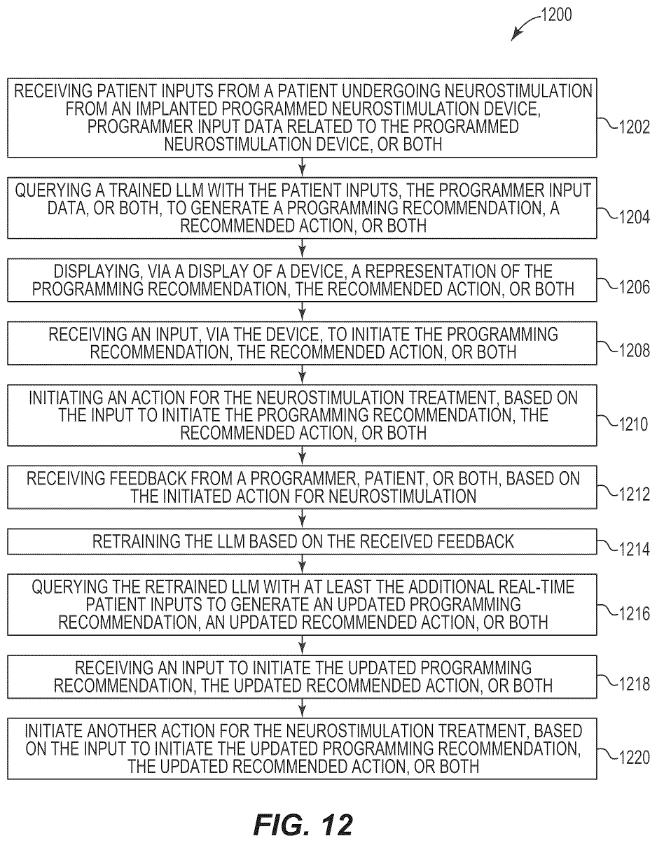

FIG. 12 illustrates an example method 1200 for large language model (LLM)-based neurostimulation programming recommendations. The method 1200 can be performed by one or more of the devices and systems herein. For instance, the method 1200 can be performed by one or more of the devices in the system 100.

At 1202, the method 1100 receiving patient inputs from a patient undergoing neurostimulation from an implanted programmed neurostimulation device, programmer input data related to the programmed neurostimulation device, or both, as described herein. At 1204, the method 1100 includes querying a trained LLM with the patient inputs, the programmer input data, or both, to generate a programming recommendation, a recommended action, or both, as described herein. At 1206, the method 1200 can include providing a representation of a programming recommendation such as displaying, via a display of a device, a representation of the recommendation, the recommended action, or both, as described herein. At 1208, the method 1200 includes receiving an input, via the device, to initiate the programming recommendation, the recommended action, or both, as described herein. At 1210, the method 1200 includes initiating an action for the neurostimulation treatment, based on the input to initiate the programming recommendation, the recommended action, or both, as described herein. The action can be altering a neurostimulation treatment applied to a patient e.g., implementing a programming recommendation and/or can include other types of actions such as prompting the patient and/or a programming with one or more questions related to the neurostimulation treatment.

Aspects of the methods herein can be performed iteratively or repeatedly, for instance, until a desired outcome (e.g., from a programmer perspective, from the patient perspective, or both) for the neurostimulation treatment of a patient is achieved. For instance, at 1212, the method 1200 can include receiving feedback from a programmer, patient, or both, based on the initiated action for neurostimulation (e.g., the application of the programming recommendation, etc.). For example, the feedback can be provided via a clinician interaction computing device and/or a patient interaction computing device. In some embodiments, the feedback can be feedback (e.g., text, audio, or visual/video feedback) that is indicative of a patient's condition responsive to application of the programming recommendation. Thus, the methods herein can readily ascertain any change and/or a degree of effectiveness of the LLM-based programming recommendations. Alternatively, or in addition, the feedback can include responses to one or more questions (e.g., LLM-based recommended actions) posed to the patient and/or the programmer.

At 1214, the method 1200 can include retraining the LLM based on the received feedback. For instance, patient and/or programmer feedback on any change and/or a degree of effectiveness of the LLM-based programming recommendation can be provided to the LLM for the purpose of retraining the LLM. Similarly, any responses to one or more questions (e.g., LLM-based recommended action) posed to the patient and/or the programmer can be provided to the LLM for the purpose of retraining the LLM. Hence, the LLM can be retrained based on real-time and/or real-world input from the perspective of the patient and/or the programmer.

At 1216, the method 1200 can include querying the retrained LLM with at least the additional real-time patient inputs to generate an updated programming recommendation, an updated recommended action, or both. For instance, the additional patient inputs (e.g., patient inputs provided via the patient interaction computing device by the neurostimulation patient in real-time) and/or additional programmer inputs (e.g., provided via the clinician interaction computing device by the programmer in real-time) can be received and can be used to query the retrained LLM and thereby generate an updated programming recommendation, an updated recommended action, or both.

Similar to 1218, the method can include receiving an input to initiate the updated programming recommendation, the updated recommended action, or both, as indicated at 1218. Responsive to the input at 1218, the method 1200 can initiate another action for the neurostimulation treatment, based on the input to initiate the updated programming recommendation, the updated recommended action, or both, as indicated at 1220. That is, the LLM can be retrained periodically or continually and can provide programming recommendations and/or recommended actions that mitigate or manage a patient's condition (e.g., improve the effectiveness of neurostimulation, particularly over time as the LLM training improves and/or programming recommendations are tailored (e.g., iteratively) to the particular patient.

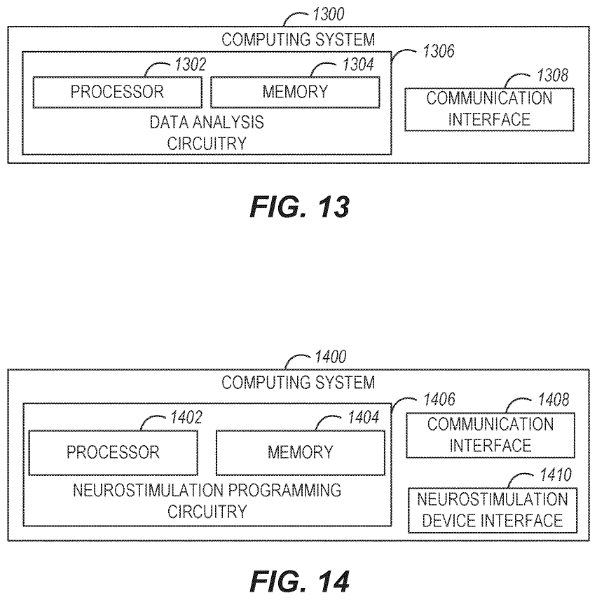

FIG. 13 illustrates, by way of example, a block diagram of an embodiment of a system 1300 (e.g., a computing system) implementing LLM-based data analysis of patient inputs to monitor, modify, or effect operation and output of a neurostimulation programming mode. The system 1300 may be integrated with or to a remote-control device, patient programmer device, clinician programmer device, program modeling system, or other external device, usable for the adjustment of neurostimulation programming. In some examples, the system 1300 may be a networked device connected via a network (or combination of networks) to a programming device or programming service using a communication interface 1308. The network may include local, short-range, or long-range networks, such as Bluetooth, cellular, IEEE 802.11 (Wi-Fi), or other wired or wireless networks.

The system 1300 includes a processor 1302 and a memory 1304, which can be optionally included as part of input and weighting control circuitry 1306. The processor 1302 may be any single processor or group of processors that act cooperatively. The memory 1304 may be any type of memory, including volatile or non-volatile memory. The memory 1304 may include instructions, which when executed by the processor 1302, cause the processor 1302 to implement the features of the user interface, or to enable other features of the input and weighting control circuitry 1306. Thus, electronic operations in the system 1300 may be performed by the processor 1302 or the circuitry 1306.

For example, the processor 1302 or circuitry 1306 may implement any of the features of the methods 1100, 1200 to obtain and process patient inputs (e.g., text, audio, and visual inputs), identify a state of a human patient and a state of the neurostimulation treatment, and initiate an action (e.g., a reprogramming recommendation) based on the state of a human patient and the state of the neurostimulation treatment.

FIG. 14 illustrates, by way of example, a block diagram of an embodiment of a system 1400 (e.g., a computing system) implementing neurostimulation programming circuitry 1406 to cause programming of an implantable electrical neurostimulation device, for accomplishing the therapy objectives in a human subject as discussed herein. The system 1400 may be operated by a clinician, a patient, a caregiver, a medical facility, a research institution, a medical device manufacturer or distributor, and embodied in a number of different computing platforms. The system 1400 may be a remote-control device, patient programmer device, program modeling system, or other external device, including a regulated device used to directly implement programming commands and modification with a neurostimulation device. In some examples, the system 1400 may be a networked device connected via a network (or combination of networks) to a computing system operating a user interface computing system using a communication interface 1408. The network may include local, short-range, or long-range networks, such as Bluetooth, cellular, IEEE 802.11 (Wi-Fi), or other wired or wireless networks.

The system 1400 includes a processor 1402 and a memory 1404, which can be optionally included as part of neurostimulation programming circuitry 1406. The processor 1402 may be any single processor or group of processors that act cooperatively. The memory 1404 may be any type of memory, including volatile or non-volatile memory. The memory 1404 may include instructions, which when executed by the processor 1402, cause the processor 1402 to implement the features of the neurostimulation programming circuitry 1406. Thus, the electronic operations in the system 1400 may be performed by the processor 1402 or the circuitry 1406.

The processor 1402 or circuitry 1406 may implement any of the features of the method 1200 (including operations 1210) to identify neurostimulation programming parameters, and implement (e.g., save, persist, activate, control) the programming parameters or relevant programs in the neurostimulation device, with use of a neurostimulation device interface 1410. The processor 1402 or circuitry 1406 may further provide data and commands to assist the processing and implementation of the programming using communication interface 1408. It will be understood that the processor 1402 or circuitry 1406 may also implement other aspects of the LLM-based processing patient input and device data processing, or device programming functionality described herein.

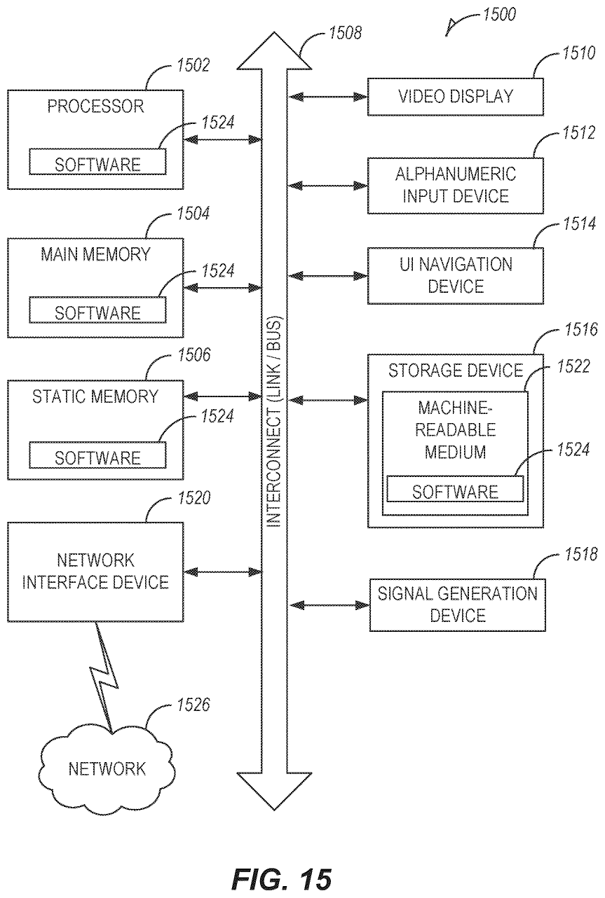

FIG. 15 is a block diagram illustrating a machine in the example form of a computer system 1500, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1500 includes at least one processor 1502 (e.g., a central processing unit (CPU), a graphics processing unit (GPU) or both, processor cores, compute nodes, etc.), a main memory 1504 and a static memory 1506, which communicate with each other via a link 1508 (e.g., bus). The computer system 1500 may further include a video display unit 1510, an alphanumeric input device 1512 (e.g., a keyboard), and a user interface (UI) navigation device 1514 (e.g., a mouse). In one embodiment, the video display unit 1510, input device 1512 and UI navigation device 1514 are incorporated into a touch screen display. The computer system 1500 may additionally include a storage device 1516 (e.g., a drive unit), a signal generation device 1518 (e.g., a speaker), a network interface device 1520, and one or more sensors (not shown), such as a global positioning system (GPS) sensor, compass, accelerometer, or another type of sensor. It will be understood that other forms of machines or apparatuses (such as PIG, RC, CP devices, and the like) that are capable of implementing the methodologies discussed in this disclosure may not incorporate or utilize every component depicted in FIG. 15 (such as a GPU, video display unit, keyboard, etc.).

The storage device 1516 includes a machine-readable medium 1522 on which is stored one or more sets of data structures and instructions 1524 (e.g., software) embodying or utilized by any one or more of the methodologies or functions described herein. The instructions 1524 may also reside, completely or at least partially, within the main memory 1504, static memory 1506, and/or within the processor 1502 during execution thereof by the computer system 1500, with the main memory 1504, static memory 1506, and the processor 1502 also constituting machine-readable media.

While the machine-readable medium 1522 is illustrated in an example embodiment to be a single medium, the term “machine-readable medium” may include a single medium or multiple media (e.g., a centralized or distributed database, and/or associated caches and servers) that store the one or more instructions 1524. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1524 may be transmitted or received over a communications network 1526 by any transmission medium via the network interface device 1520 using any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

Terms used herein should be accorded with their ordinary meaning in the relevant arts, or the meaning indicated by their use in context, but if an express definition is provided, that meaning controls.

Herein, references to “one embodiment”, “an embodiment”, “one implementation”, or “an implementation” do not necessarily refer to the same embodiment or implementation, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those with skill in the relevant art(s).

What is claimed is:

1. A method for generating large language model (LLM)-based neurostimulation programming recommendations, the method comprising:
receiving patient inputs from a patient undergoing neurostimulation from a programmed neurostimulation device, programmer input data related to the programmed neurostimulation device, or both;
querying a trained LLM with the patient inputs, the programmer input data, or both, to generate a programming recommendation, a recommended action, or both;
providing, via a device, a representation of the programming recommendation, the recommended action, or both, wherein the device is a clinician interaction computing device, a patient interaction computing device, or both;
receiving an input, via the device, to initiate the programming recommendation, the recommended action, or both; and
responsive to receipt of the input, initiating an action for the neurostimulation treatment, based on the input to initiate the programming recommendation, the recommended action, or both.

2. The method of claim 1, wherein the input comprises an input to initiate the programming recommendation; and wherein initiating the action comprises applying the programming recommendation to the programmed neurostimulation device.

3. The method of claim 2, further comprising:
receiving patient feedback associated with the applied programming recommendation, receiving programmer feedback associated with the applied programming recommendation, or both; and
retraining the trained LLM based on the received patient feedback associated with the applied programming recommendation, the programmer feedback associated with the applied programming recommendation, or both.

4. The method of claim 3, further comprising:
receiving updated programmer input data, updated patient input, or both;
querying the retrained LLM with the updated programmer input data, the updated patient input, or both, to generate an updated programming recommendation, an updated recommended action, or both;
providing, via the device, the updated programming recommendation, the updated recommended action, or both;
receiving an input to initiate the updated programming recommendation, the updated recommended action, or both; and
initiating an updated action for the neurostimulation treatment, based on the input to initiate the updated programming recommendation, the updated recommended action, or both.

5. The method of claim 1, further comprising training a LLM to yield the trained LLM, wherein the LLM is trained with at least neuromodulation subject matter expert input.

6. The method of claim 1, wherein the patient inputs are provided during a communication session with a chatbot, during a communication session with a programmer, or both.

7. The method of claim 1, further comprising retraining the trained LLM with the patient inputs from a patient undergoing neurostimulation, the programmer input data related to the programmed neurostimulation device, or both.

8. The method of claim 1, further comprising retraining the trained LLM based on programmer-patient interaction data obtained during a programmer-patient interaction, wherein the programmer-patient interaction data further comprises voice-to-text data, voice-to-voice data, and/or visual data associated with the patient obtained during the programmer-patient interaction.

9. The method of claim 1, further comprising generating, via the trained LLM, a programming recommendation, wherein the programming recommendation is based on predefined neurostimulation parameters in the trained LLM, the predefined neurostimulation parameters including:
available programming settings for the programmed neurostimulation device;
clinician approved programming settings for the programmed neurostimulation device;
previously utilized programming settings for the programmed neurostimulation device; or
any combination thereof.

10. The method of claim 1, wherein the programming recommendation further comprises a recommendation to modify a parameter of a currently applied neurostimulation program to the programmed neurostimulation device, and wherein initiating the action for the neurostimulation treatment further comprises providing the programming recommendation to modify a parameter to a clinician interaction computing device, a patient interaction computing device, or both.

11. The method of claim 1, wherein the programming recommendation further comprises a recommendation to apply a different neurostimulation program to the programmed neurostimulation device than a currently applied neurostimulation program to the programmed neurostimulation device, and wherein initiating the action for the neurostimulation treatment further comprises providing the programming recommendation to apply the different neurostimulation program to a clinician interaction computing device, a patient interaction computing device, or both.

12. The method of claim 1, wherein initiating the action for the neurostimulation treatment further comprises providing a question to the patient related to the neurostimulation treatment, providing a question to the programmer related to the neurostimulation treatment, or both.

13. The method of claim 12, further comprising:
receiving a response to the question from the patient, the question to the programmer, or both; and
retraining the LLM based on the received response.

14. The method of claim 1, wherein the trained LLM further comprises a trained LLM that is trained at least on a data set that includes training on a plurality of past patient interactions that are local to the patient.

15. The method of claim 1, wherein querying the trained LLM generates both the programming recommendation and the recommended action, and wherein the recommended action further comprises providing a notification provided to the patient that is indicative at least of a scheduled time of application of the programming recommendation to the programmed neurostimulation device.

16. A non-transitory computer-readable medium comprising instructions that, when executed by a processor, cause the processor to:
receive two or more different types of real-time patient inputs from the patient that are related to the programmed neurostimulation device and programmer input data related to the programmed neurostimulation device, wherein the two or more different types of real-time patient inputs include two or more of text inputs, audio inputs, and visual inputs;
query a trained large language model (LLM) with the plurality of different types of real-time patient inputs or a derivative of the plurality of real-time patient inputs and programmer input data, to generate a programming recommendation, a recommended action, or both;
display, via a display of a device, a representation of the programming recommendation, the recommended action, or both;
receive input to initiate the programming recommendation, the recommended action, or both; and
responsive to receipt of the input, initiate an action for the neurostimulation treatment, based on the input to initiate the programming recommendation, the recommended action, or both.

17. The medium of claim 16, wherein the query of the trained LLM generates both the programming recommendation and the recommended action, and wherein the recommended action further comprises providing a notification to the patient that is indicative of a scheduled time of application the programming recommendation to the programmed neurostimulation device, details associated with the programming recommendation, or both, and wherein the processor is further configured to provide the notification to the patient.

18. A system for generating large language model (LLM)-based neurostimulation programming recommendations, the system comprising:
a programmed neurostimulation device implanted in a patient;
a large language model (LLM) trained with expert knowledge in neurostimulation programming; and
a processor configured to:
receive i) patient inputs from the patient that are related to the programmed neurostimulation device, ii) programmer input data related to the programmed neurostimulation device, or both i) and ii);
query a trained large language model (LLM) with the patient inputs, the programmer input data, or both, to generate a programming recommendation, a recommended action, or both;
display, via a display of a device, a representation of the programming recommendation, the recommended action, or both;
receive input to initiate the programming recommendation, the recommended action, or both;
responsive to receipt of the input, initiate an action for the neurostimulation treatment, based on the input to initiate the programming recommendation, the recommended action, or both;
receive feedback from a programmer, patient, or both, based on the initiated action for neurostimulation;
retrain the LLM based on the received feedback;
query the retrained LLM with at least the additional patient inputs to generate an updated programming recommendation, an updated recommended action, or both;
receive input to initiate the updated programming recommendation, the updated recommended action, or both; and
initiate another action for the neurostimulation treatment, based on the input to initiate the updated programming recommendation, the updated recommended action, or both.

19. The system of claim 18, wherein the programmed neurostimulation device is a spinal cord stimulation device.

20. The system of claim 18, wherein the programmed neurostimulation device is a deep brain stimulation device.
